(19) United States
(12) Patent Application Publication
(10) Pub. No.: US 2005/0096254 A1
(43) Pub. Date: May 5, 2005

(54) Peptide deformylase activated prodrugs

(76) Inventors: Maria V Sergeeva, San Diego, CA (US); Venkata Ramana Doppalapudi, San Diego, CA (US)

Correspondence Address: Antoinette F Konski;Bingham McCutchen, 18th Floor, Three Embarcadero Center, San Francisco, CA, 94111-4067, US

(21) Appl. No.: 10/511,489

(22) PCT Filed: Apr. 17, 2003
(86) PCT No.: PCT/US03/11981

(30) Foreign Application Priority Data
Apr. 18, 2002 (US) 60374089

(51) Int. Cl. A61K 38/17

(52) U.S. Cl. 514/2; 530/409

(57) ABSTRACT

This invention provides a method for inhibiting the growth of a microorganism that expresses Peptide Deformylase by contacting the microorganism with an effective amount of the compound described herein. This method inhibits the growth of gram-positive and gram-negative microorganism, e.g., S. aureus, S. epidermidis, K. pneumoniae, E. aerogenes, and E. cloacae. This method can be practiced in vitro, ex vivo and in vivo. Further provided is a method for alleviating the symptoms of an infection by a Peptide Deformylase expressing microorganism in a subject by administering or delivering to the subject an effective amount of the compound described above.

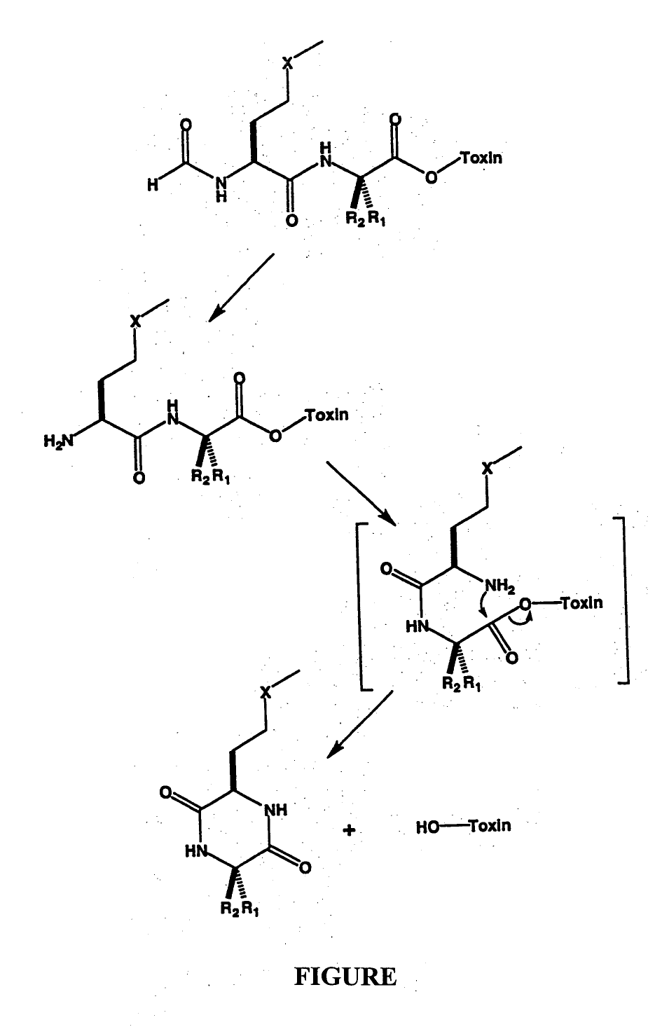

### CROSS-REFERENCE TO RELATED APPLICATIONS

This application claims priority under 35 U.S.C. § 119 (e) to U.S. Provisional Application Ser. No. 60/374,089, filed Apr. 18, 2002, the contents of which are hereby incorporated by reference into the present disclosure.

### TECHNICAL FIELD

The present invention relates to the field of Enzyme Catalyzed Therapeutic Activation (ECTA™) therapy and in particular, ECTA therapies specific for microorganisms that express Peptide Deformylase (“PDF”).

### BACKGROUND

Throughout this disclosure, various publications are referenced by first author and date, within parentheses, patent number or publication number. The complete bibliographic reference is given at the end of the application. The disclosures of these references are hereby incorporated by reference into this disclosure to more fully describe the state of the art to which this application pertains.

Enzyme Catalyzed Therapeutic Activation (ECTA™) therapy is a novel technology that provides unique prodrug substrates for target enzymes. Unlike conventional therapies, ECTA prodrugs neither inhibit nor irreversibly inactivate the target enzyme. U.S. Pat. No. 6,159,706; PCT/US98/16607; PCT/US99/01332; and PCT/US00/20008.

Target enzymes convert the ECTA prodrug into a toxin preferentially within the target cell or in an environment wherein the target enzyme is expressed as compared to an environment where it is absent, as in an infected cell. Because the compounds do not require a targeting agent, they can be directly utilized, topically or systemically.

ECTA molecules do not, in most instances, yield cytotoxic products spontaneously (without target enzyme activation). They are not be appreciably activated by non-targeted enzymes, as-this may result in toxicity to non-diseased or non-infected tissue. Table 1 summarizes the characteristics of ECTA molecules and enzyme activators.

TABLE 1

| Characteristics of ECTA Target | |
|---|---|
| Enzymes | Characteristics of ECTA Prodrugs |
| Infectious Disease: Must be | Must be able to get into cells (by |
| present only in target cells | itself or as prodrug). |
| (including diseased cells, | |
| bacteria, fungi, etc.). The | |
| enzyme should be necessary | |
| for continued viability or | |
| pathogenicity. | |
| Must process a molecule | At least one of the |
| that resembles the natural | products formed from |
| substrate (an ECTA molecule) | the enzymatic reaction |
| into cytotoxic species. The | must be cytotoxic. However, |
| resemblance only has to be | the ECTA remain in inactive |
| significant with respect to the | form until activated by the |
| specificity of the enzyme/substrate | target enzyme. The |
| interaction and the ability of the | compound must have a |
| enzyme to process the substrate | high degree of specificity for the |
| intracellularly into toxic species. | targeted enzyme, although |
| | conversion by a non-targeted |
| | enzyme is acceptable |
| | if the product(s) are |
| | not cytotoxic. |
| Must not be inactivated by | Must not inhibit or deactivate |
| the ECTA molecule, | the targeted enzyme. |
| intermediate(s), or the | |
| product(s) of the reaction. | |

In cases of bacterial, viral and fungal infections in plants, people or agriculturally important animals, metabolic pathways being present in the pathogenic organisms, but absent in the host are a source of potential ECTA target enzymes. For example, some pathways, as well as the enzymes involved, have only been found in bacteria, fungi and plants and not in mammalian cells. One example is the synthesis of “essential” amino acids—amino acids that animals cannot synthesize and must ingest with food. Nelson and Cox (1972).

Another example is Peptide Deformylase (“PDF”, EC 3.5.1.31) which catalyses deformylation of N-terminal N-formyl methionine in a growing polypeptide chain. Meinnel (1999). The enzyme is present and active in bacteria (Meinnel, et al, 1993), but has not been reported to be present in mammalian cells. Sequences homologous to bacterial PDF sequences have been recently found in mammals but their exact function is unknown. Giglione, et al. (2000a) and (2000b).

Because the enzyme is not active in humans it has been used as a target for antibacterial drugs, mostly PDF inhibitors. Dithiols can act as non-specific PDF inhibitors by coordination of sulfhydryl groups with the active site metal ion. Rajagopalan, et al. (1997). In case of 1,2- or 1,3-dithiols a slow extraction of the metal ion from the active site takes place. The formation of stable 5- or 6-membered rings, respectively, each containing two metal-sulfur bonds, accounts for this effect.

A rationally designed combinatorial library was used to select mechanism-based PDF inhibitors of the general structure HS—CH₂—CH(Rₐ)—CONH—CH(R_{b})—CONH-R_{c}. Wei et al. (2000). The optimal inhibitor selected from the library possesses an n-Bu group as an Rₐ, R_{b}=—(CH₂)₃—NH—C(═NH)—NH₂, and R_{c} is 2-naphthalene. This compound acts as a competitive PDF inhibitor with a Kᵢ of 15 nM.

Jayasekera, et al. (2000) describes a series of non-peptidic compounds structurally related to the known anticholesteremic thyropropic acid to inhibit E. coli PDF. Actinonin is reported to be a potent PDF inhibitor with activity in the subnanomolar Kᵢ range. Chen, et al. (2000).

Wei and Pei (2000) describe that 5′-dipeptidyl derivatives of 5-fluorodeoxyuridine release a small molecule (5-fluorodeoxyuridine (5-F-dUrd)) upon PDF catalyzed deformylation. 5-F-dUrd formation was monitored in the reaction of the substrate catalyzed by purified PDF or E. coli crude lysates. The compound was marginally cytotoxic (IC₅₀>100, when applied to E. coli bacteria. Potency was not increased by increased expression of PDF in bacteria (using a PDF-overexpressing strain). The compound was slightly more effective (IC₅₀=50 μM) against gram-positive microorgansims.

Additional inhibitors are described in Apfel et al. (2000), Apfel et al. (2001a), Apfel et al. (2001b), Clements et al. (2001), Durand et al. (1999), and Chen et al. (2000). However, a compound or agent that is selectively and effectively activated by PDF to a toxin has not been described. This invention satisfies this need and provides related advantages as well.

### DISCLOSURE OF THE INVENTION

Thus, in one aspect, the invention provides a prodrug compound having the structure: 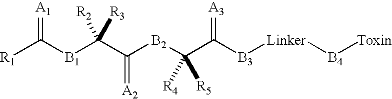
wherein the toxin is a cytotoxic or antibiotic molecule that is released upon activation by an enzyme, other than 5-F-dUrd;
wherein R₁, R₂, R₄, and R₅ are independently the same or different and are selected from the group consisting of hydrogen, a substituted or unsubstituted C₅-C₁₄ aromatic or heteroaromatic (for example: phenylmethylene, 4-hydroxyphenylmethylene, imidazolemethylene, etc.); and a substituted or unsubstituted saturated or unsaturated C₁-C₆ alkyl (for example: methyl, ethyl, 3-hydroxypropyl, 3-aminopropyl, N-methyl-3-aminoethyl, 2-methoxyethyl, etc.);
wherein R₃ is selected from the group consisting of a substituted or unsubstituted aromatic or heteroaromatic (for example: phenylmethylene; triazolemethylene, thiophenemethylene, etc.), and a substituted or unsubstituted saturated or unsaturated C₁-C₆ alkyl (for example: ethyl, propyl, 2-hydroxyethyl, etc.) and —CH₂—CH₂—X—CH₃, wherein X is selected from the group consisting of O, S, NH, NR₆, and CH₂; where R₆ is a lower alkyl such as, for example, methyl or ethyl;
wherein A₁ and A₃ are independently the same or different and are selected from the group consisting of ═O, ═S, ═NH, ═N—OH, or ═N—R₇, where R₇ is hydrogen or a C₁-C₆ alkyl such as, for example, methyl, ethyl, or methoxymethyl;
wherein A₂ is selected from the group consisting of ═O, ═S; ═NH, ═N—OH, ═N—R₈, or ═C(R₉)(R₁₀), wherein R₈, R₉, and R₁₀ are independently the same or different and are selected from the group consisting of hydrogen or a C₁-C₆ alkyl such as, for example, methyl, ethyl, or methoxymethyl;
wherein B₁ is selected from the group consisting of —O—, —S—, —NH— or —N(R₁₁)—, wherein R₁₁ is selected from the group consisting of hydrogen and a C₁-C₆ alkyl such as, for example, methyl, ethyl, or methoxymethyl;
wherein B₂ is absent or is selected from the group consisting of —O—, —S—, —N(R₁₂)—, or —C(R₁₃)(R₁₄)—, where R₁₂, R₁₃, and R₁₄ are independently the same or different and are selected from the group consisting of hydrogen or a substituted or unsubstituted saturated or unsaturated C₁-C₆ alkyl (for example: methyl, ethyl, 3-r hydroxypropyl, 3-aminopropyl, N-methyl-3-aminoethyl, 2-methoxyethyl, etc.), wherein when B₂ is —N(R₁₂)— or —C(R₁₃)(R₁₄)— it can be additionally joined through R₁₂, R₁₃ or R₁₄ to R₄ or R₅ to form a cyclic structure; wherein the fragment —B₂—C(R₄)(R₅)—C(═A₃)— in its entirety is proline or a proline derivative or analog,
wherein B₃ is absent or is selected from the group consisting of —O—, —S—, or —NH—, or —N(R₁₅—, wherein R₁₅ is selected from the group consisting of hydrogen and a C₁-C₆ alkyl such as, for example, methyl, ethyl, or methoxymethyl;
wherein B₄ is absent or is selected from the group consisting of —O—, —S—, —N(R₆)—, and —C(R₁₆)(R₁₇)— and wherein R₁₆ and R₁₇ are independently the same or different and are selected from the group consisting of hydrogen or a substituted or unsubstituted saturated or unsaturated C₁-C₆ alkyl such as, for example, methyl, ethyl, or methoxymethyl;
wherein a Linker is absent or is a traceless linker and may be selected from one of the following structures: 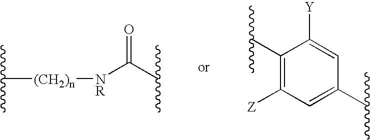
wherein n=2 or 3 and R is a lower alkyl such as, for example, methyl or ethyl;
wherein Y and Z are independently the same or different and are selected from the group consisting of hydrogen, lower alkyl, substituted or unsubstituted lower alkenyl, substituted or unsubstituted lower alkynyl, substituted or unsubstituted aryl groups, substituted or unsubstituted heterocyclic groups, substituted or unsubstituted lower alkoxy, lower alkylthio, halogen, cyano, nitro, carboxylate, sulfonate, alkyl sulfone, alkylsulfoxide and trialkylsilyl.

In one aspect, wherein R₁ and R₂ are independently the same or different and are selected from the group consisting of a substituted or unsubstituted C₁ to C₆ lower alkyl. In a further aspect, R₁ and R₂ are independently the same or different and are selected from the group consisting of methyl and H. In yet a further aspect, R₁ and R₂ each are H.

In one aspect, R₃ is —CH₂—CH₂—X—CH₃, wherein X is selected from the group consisting of oxygen, sulfur or methylene. In a further aspect, R₄ is selected from the group consisting of a substituted or unsubstituted, saturated or unsaturated C₁ to C₆ lower alkyl and H. In a yet further aspect, R₄ is methyl or H. Also provided is a compound wherein R₄ and R₅ are independently the same or different and are selected from the group consisting of H and a substituted or unsubstituted C₁ to C₆ alkyl.

In another aspect, R₄ and R₅ are independently the same or different and are selected from the group consisting of H and methyl. In an alternative embodiment, either or both of R₄ and R₅ are H.

In one aspect, the peptide deformylase ECTA compounds have the structure of N-formyl-Met-Leu-linker-prototoxophore.

In one aspect, the compound has the structure:

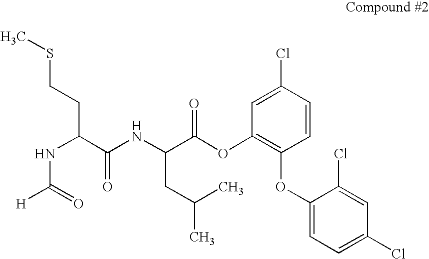

In one aspect, the compound has the structure:

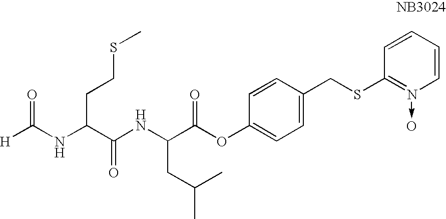

In one aspect, the compound has the structure:

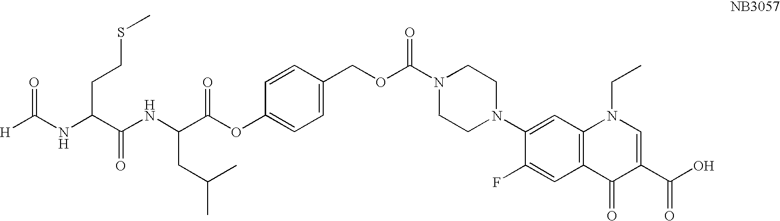

In one aspect, the compound has the structure:

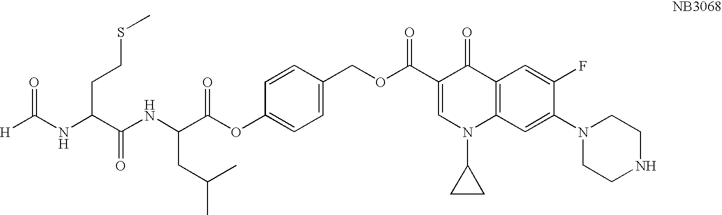

In one aspect, the compound has the structure:

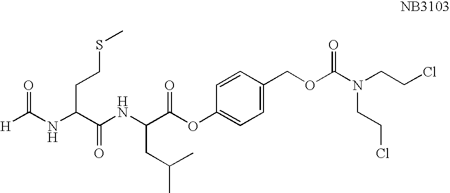

In one aspect, the compound has the structure:

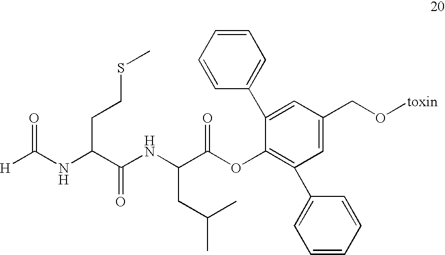

When toxin is absent, compound is NB3145.

In one aspect, the compound has the structure:

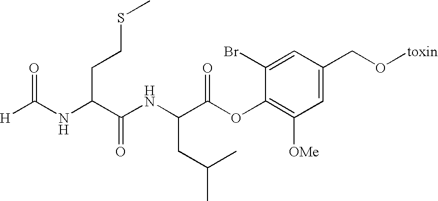

When toxin is absent, compound is N3162.

In one aspect, the compound has the structure:

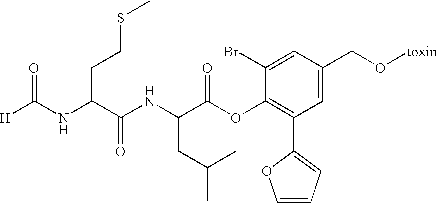

When toxin is absent, compound is NB3177.

In one aspect, the compound has the structure:

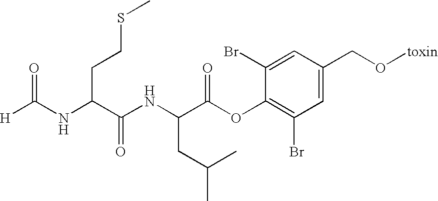

When toxin is absent, compound is NB3144. In one aspect, the compound has the structure:

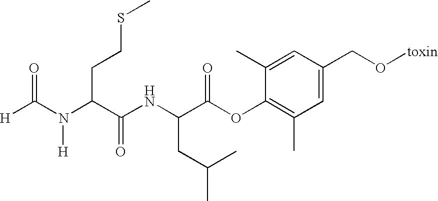

When toxin is absent, compound is NB3165.

Also provided by this invention is a method for inhibiting the growth of a microorganism that expresses PDF by contacting the ism with an effective amount of the compound as describe above. This method inhibits the growth of gram-positive and gram-negative microorganism, e.g., S. aureus, S. epidermidis, K. pneumoniae, E. aerogenes, and E. cloacae. This method can be practiced in vitro, ex vivo and in vivo. Further provided is a method for alleviating the symptoms of an infection by a PDF expressing microorganism in a subject by administering or delivering to the subject an effective amount of the compound described above. A “subject” is defined herein and includes mammals such as human patients.

This invention also provides a composition comprising the prodrug compounds as described above, alone or in combination with other compounds or other agents, known or yet to be discovered, and a carrier. In one aspect, the carrier is another molecule or an inert substance such as a plate or column. In an alternative embodiment, the carrier is a pharmaceutically acceptable carrier. Pharmaceutically acceptable carriers are known in the art and described briefly above.

### BRIEF DESCRIPTION OF THE FIGURE

The FIGURE proposes a reaction scheme for PDF activation of the compounds of this invention.

### MODES FOR CARRYING OUT THE INVENTION

As used herein, certain terms may have the following defined meanings.

The singular form “a,” “an” and “the” include plural references unless the context clearly dictates otherwise. For example, the term “a cell” includes a plurality of cells, including mixtures thereof.

The term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “lower alkyl, alkynyl, or alkenyl” means a straight, branched or cyclic group and unless otherwise defined, containing between one and ten carbons (a C₁-C₁₀), or alternatively a C₁-C₆, or alternatively a C₁-C₄-containing group.

As used herein the term “prodrug” means a precursor or derivative form of a pharmaceutically active agent or substance that is less cytotoxic to a target cell as compared to the drug metabolite and is capable of being enzymatically activated or converted into the more active form.

A “composition” is intended to mean a combination of active agent and another compound or composition, inert (for example, a surface, a paint, a detectable agent or label or a pharmaceutically acceptable carrier) or active, such as an adjuvant or disinfectant.

A “pharmaceutical composition” is intended to include the combination of an active agent with a carrier, inert or active, making the composition suitable for diagnostic or therapeutic use in vitro, in vivo or ex vivo.

The term “prophylactically effective amount” refers to an amount effective in preventing infection in a subject or plant infestation.

The term “pharmaceutically acceptable carrier” and “biologically acceptable carrier” refer to a carrier or adjuvant that is administered to a host or patient, together with a compound of this invention, and which does not destroy the pharmacological activity thereof and is non-toxic, when administered in doses sufficient to deliver an effective amount of the compound. Examples of suitable carriers include liquid phase carriers, such as sterile or aqueous solutions, as well as those described below. Examples of pharmaceutically acceptable carrier include any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

A “substituent” refers to a group that replaces one or more hydrogens attached to a carbon or nitrogen in a substituted group. Exemplary substituents include alkyl, alkylidenyl, alkylcarboxy, alkoxy, alkenyl, alkenylcarboxy, alkenyloxy, aryl, aryloxy, alkylaryl, alkylaryloxy, —OH, amide, carboxamide, carboxy, sulfonyl, ═O, ═S, —NO₂, halogen, haloalkyl, fused saturated or unsaturated optionally substituted rings, —S(O)R, —SO₃R, —SR, —NRR′, —OH, —CN, —C(O)R, —OC(O)R, —NHC(O)R, —(CH2)ₙCO₂R or —(CH2)ₙCONRR′ where n is 0-4, and wherein R and R′ are independently H, alkyl, aryl or alkylaryl. Substituents also include replacement of a carbon atom and one or more associated hydrogen atoms with an optionally substituted heteroatom.

The term “treating” refers to any of the following: the alleviation of symptoms of a particular disorder in a patient; the improvement of an ascertainable measurement associated with a particular disorder; or a reduction in microbial number. One of skill in the art can determine when a host has been “treated” by noting a reduction in microbial load or an alleviation in symptoms associated with infection.

The term “pharmaceutically acceptable salt, prodrug or derivative” relates to any pharmaceutically acceptable salt, ester, ether, salt of an ester, solvate, such as ethanolate, or other derivative of a compound of the present invention which, upon administration to a recipient, is capable of providing (directly or indirectly) a compound of this invention or an active metabolite or residue thereof. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system).

Salts of the compounds of the present invention may be derived from inorganic or organic acids and bases. Examples of acids include hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acids. Other acids, such as oxalic, while not in themselves pharmaceutically acceptable, may be employed in the preparation of salts useful as intermediates in obtaining the compounds of the invention and their pharmaceutically acceptable acid addition salts. Examples of bases include alkali metal (e.g., sodium) hydroxides, alkaline earth metal (e.g. magnesium) hydroxides, ammonia, compounds of formula NW₄⁺, wherein W is C₁₋₄ alkyl and THAM (2-amino-2-hydroxymethyl-1,3-propanediol).

Examples of salts include: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate; persulfate, phenylproprionate, picrate, pivalate, propionate, succinate, tartrate, thiocyanate, tosylate and undecanoate. Other examples of salts include anions of the compounds of the present invention compounded with a suitable cation such as Na⁺, Li⁺, NH₄⁺, and NW₄⁺ (wherein W is a C₁₋₄ alkyl group).

For therapeutic use, salts of the compounds of the present invention will be pharmaceutically acceptable. However, salts of acids and bases which are non-pharmaceutically acceptable may also find use, for example, in the preparation or purification of a pharmaceutically acceptable compound or for use to reduce microbial infestation in plants.

The term “traceless Linker” indicates a spacer or connector between two parts of a single molecule such that when a particular bond is severed between the two parts of the molecule, the connector which is still attached to the second part of the molecule, eliminates leaving no trace of itself. See, for example, F.M.H. de Groot et al. (2000) J. Med. Chem. 43:3093-3102.

The term “effective amount” is to include therapeutically or prophylactically effective amounts. The term refers to an amount effective in treating or preventing an infection in a patient or an infestation in a plant either as monotherapy or in combination with other agents.

“Inhibiting the growth” of a microorganism means reducing by contact with an agent, the rate of proliferation of such a microorganism, in comparison with a control microorganism of the same species not contacted with this agent.

A “subject” is any living being that is or can be a direct or indirect host to a PDF expressing microorganism, including plants and animals such as a fish, an avian or a mammal, and preferably a human. Fish include, but are not limited to pets and aquaculture. Avians include, but are not limited to pets, sport animals and farm animals. Mammals include, but are not limited to, murines, simians, humans, farm animals, sport animals, and pets.

Examples include, but are not limited to non-vertebrates, vertebrates, e.g., avians or mammals, such as human patients. Mammals include, but are not limited to, murines, simians, humans, farm animals, sport animals, and pets.

PDF is a well-studied enzyme. The crystallographic structure of it is known. Chan et al. (1997). The enzyme has been expressed in E. coli BL21(DE3) cells Rajagopalan et al. (1997). The authors of the paper isolated the E. coli def gene by PCR using the primers designed based on the literature data on the sequence of the gene. Purified enzyme is unstable due to fast oxidation the catalytic site Fe²⁺ by the atmospheric oxygen. Rajagopalan et al. (1998). The conditions for proper handling the enzyme to avoid inactivation have been reported. Rajagopalan et al. (1997). Importantly, Zn²⁺ and Ni²⁺ containing PDF's are stable allowing for the in vitro evaluation of the enzyme catalytic properties. There exists a simple continuous colorimetric assay for PDF. Wei and Pei (1997). It utilizes N-formylmethionylleucine p-nitroanilide as a substrate. A coupled aminopeptidase reaction that follows the PDF reaction releases p-nitroaniline that can be monitored spectrophotometrically at 405 nm.

PDF is a perfect ECTA target enzyme. It is active in bacteria and inactive in human hosts. It has broad substrate specificity. Deformylation liberates a free amino group of methionine (or another amino acid tolerated in PI position of the substrate, such as norleucine) which can perform a subsequent nucleophilic attack. With a rationally designed dipeptide the free amino group can attack an optimally positioned carbonyl group of a dipeptide thus forming a cyclic molecule (diketopiperazine, DKP) originated from the dipeptide and releasing a toxin. The dipeptide can be optimized to enhance DKP formation. The scheme of the proposed reaction is given in the Figure. Here X can be sulfur (methionine) or —CH₂— (norleucine). R₁ and R₂ are aliphatic radicals that can be selected based on the published SAR data for PDF. Hu et al. (1998).

Thus, in one aspect, the invention provides a prodrug compound having the structure: 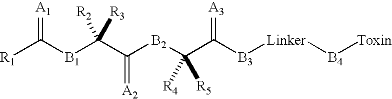
wherein the toxin is a cytotoxic or antibiotic molecule that is released upon activation by an enzyme, other than 5-F-dUrd;
wherein R₁, R₂, R₄, and R₅ are independently the same or different and are selected from the group consisting of hydrogen, a substituted or unsubstituted C₅-C₁₄ aromatic or heteroaromatic (for example: phenylmethylene, 4-hydroxyphenylmethylene, imidazolemethylene, etc.); and a substituted or unsubstituted saturated or unsaturated C₁-C₆ alkyl (for example: methyl, ethyl, 3-hydroxypropyl, 3-aminopropyl, N-methyl-3-aminoethyl, 2-methoxyethyl, etc.);
wherein R₃ is selected from the group consisting of a substituted or unsubstituted aromatic or heteroaromatic (for example: phenylmethylene; triazolemethylene, thiophenemethylene, etc.), and a substituted or unsubstituted saturated or unsaturated C₁-C₆ alkyl (for example: ethyl, propyl, 2-hydroxyethyl, etc.) and —CH₂—CH₂—X—CH₃, wherein X is selected from the group consisting of O, S, NH, NR₆, and CH₂; where R₆ is a lower alkyl such as, for example, methyl or ethyl;
wherein A₁ and A₃ are independently the same or different and are selected from the group consisting of ═O, ═S, ═NH, ═N—OH, or ═N—R₇, where R₇ is hydrogen or a C₁-C₆ alkyl such as, for example, methyl, ethyl, or methoxymethyl;
wherein A₂ is selected from the group consisting of ═O, ═S; ═NH, ═N—OH, ═N—R₈, or ═C(R₉)(R₁₀), wherein R₈, R₉, and R₁₀ are independently the same or different and are selected from the group consisting of hydrogen or a C₁-C₆ alkyl such as, for example, methyl, ethyl, or methoxymethyl;
wherein B₁ is selected from the group consisting of —O—, —S—, —NH— or —N(R₁₁), wherein R₁₁ is selected from the group consisting of hydrogen and a C₁-C₆ alkyl such as, for example, methyl, ethyl, or methoxymethyl;
wherein B₂ is absent or is selected from the group consisting of —O—S—, —N(R₁₂)—, or —C(R₁₃)(R₁₄)—, where R₁₂, R₁₃, and R₁₄ are independently the same or different and are selected from the group consisting of hydrogen or a substituted or unsubstituted saturated or unsaturated C₁-C₆ alkyl (for example: methyl, ethyl, 3-hydroxypropyl, 3-aminopropyl, N-methyl-3-aminoethyl, 2-methoxyethyl, etc.), wherein when B₂ is —N(R₁₂)— or —C(R₁₃)(R₁₄)— it can be additionally joined through R₁₂, R₁₃ or R₁₄ to R₄ or R₅ to form a cyclic structure; wherein the fragment —B₂—C(R₄)(R₅)—C(═A₃)— in its entirety is proline or a proline derivative or analog,
wherein B₃ is absent or is selected from the group consisting of —O—, —S—, or —NH—, or —N(R₁₅, wherein R₁₅ is selected from the group consisting of hydrogen and a C₁-C₆ alkyl such as, for example, methyl, ethyl, or methoxymethyl;
wherein B₄ is absent or is selected from the group consisting of —O—, —S—, —N(R₆)—, and —C(R₁₆)(R₁₇)— and wherein R₁₆ and R₁₇ are independently the same or different and are selected from the group consisting of hydrogen or a substituted or unsubstituted saturated or unsaturated C₁-C₆ alkyl such as, for example, methyl, ethyl, or methoxymethyl;
wherein a Linker is absent or is a traceless linker and may be selected from one of the following structures: 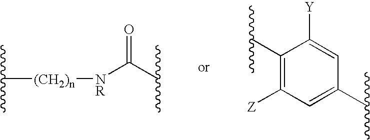
wherein n=2 or 3 and R is a lower alkyl such as, for example, methyl or ethyl;
wherein Y and Z are independently the same or different and are selected from the group consisting of hydrogen, lower alkyl, substituted or unsubstituted lower alkenyl, substituted or unsubstituted lower alkynyl, substituted or unsubstituted aryl groups, substituted or unsubstituted heterocyclic groups, substituted or unsubstituted lower alkoxy, lower alkylthio, halogen, cyano, nitro, carboxylate, sulfonate, alkyl sulfone, alkylsulfoxide and trialkylsilyl.

In one aspect, wherein R₁ and R₂ are independently the same or different and are selected from the group consisting of a substituted or unsubstituted C₁ to C₆ lower alkyl. In a further aspect, R₁ and R₂ are independently the same or different and are selected from the group consisting of methyl and H. In yet a further aspect, R₁ and R₂ are each H.

In one aspect, R₃ is —CH₂—CH₂—X—CH₃, wherein X is selected from the group consisting of oxygen, sulfur or methylene. In a further aspect, R₄ is selected from the group consisting of a substituted or unsubstituted, saturated or unsaturated C₁ to C₆ lower alkyl and H. In a yet further aspect, R₄ is methyl or H. In one aspect, the R₄ and R₅ are independently the same or different and are selected from the group consisting of H and a substituted or unsubstituted C₁ to C₆ alkyl.

In another aspect, R₄ and R₅ are independently the same or different and are selected from the group consisting of H and methyl. In an alternative embodiment, either or both of R₄ and R₅ are H.

Examples of toxins include, but are not limited to a group consisting of anthracyclins, vinca alkaloids, mitomycins, bleomycins, penicillins, cephalosporins, oxacillins, carbopenems, tetracyclins, chloramphenicols, macrolides, cycloserines, fluoroquinolones, glycopeptides, aminoglycosides, peptide antibiotics, oxazolidinones, quinolones, sulfonamides, cytotoxic nucleosides, pteridine family, nitrogen mustards, polyhalogenated biphenyls, diynenes, podophillotoxins, taxoids, doxorubicin, carminomycin, daunorubicin, aminopterin, methotrexate, methopterin, dichloromethotrexate, mitomycin C, porfiromycin, 6-mercaptopurine, cytosine arabinoside, podophillotoxin, etoposide, etoposide phosphate, melphalan, vindesine, vinblastine, vincristine, leurosidine, leurosine, bis-(2-chloroethyl)amine, trichlorcarban, trichlorocarbanilide, tribromosalicylanilide, sulphamethoxazole, chloramphenicol, cycloserine, trimethoprim, chlorhexidine, hexachlorophene, fentichlor, 5-chloro-2-(2,4-dichlorophenoxy)phenol, 4-chloro-2-(2,4-dichlorophenoxy)phenol, 3-chloro-2-(2,4-dichlorophenoxy)phenol, 6-chloro-2-(2,4-dichlorophenoxy)phenol, 5-chloro-2-(3,4-dichlorophenoxy)phenol, 5-chloro-2-(2,5-dichlorophenoxy)phenol, 5-chloro-2-(3,5-dichlorophenoxy)phenol, 2,2′-dihydroxy biphenyl ether, halogeneted 2-hydroxybenzophenones, 2-mercaptopyridine-N-oxide, combretastatin, camptothesin, apoptolidene, cisplatin, epothilone, halichondrin, hemiasterlin, methioprim, thapsigargin, chloroquine, 4-hydroxycyclophosphamide, etoposide, colchicine, melphalan, quercetin, genistein, erbstatin, N-(4-aminobutyl)-5-chloro-2-naphtalen-sulfonamide, pyridnyloxazol-2-one, isoquinolyloxazolone-2-one, verapamil, quinine, quinidine, and chloroquine.

In one aspect, the compound has the structure:

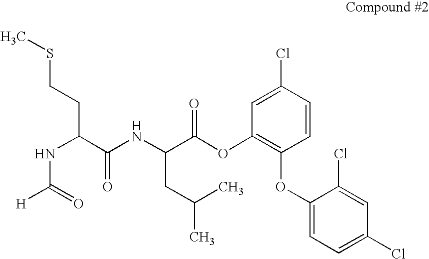

In one aspect, the compound has the structure:

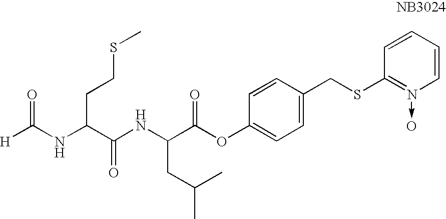

In one aspect, the compound has the structure:

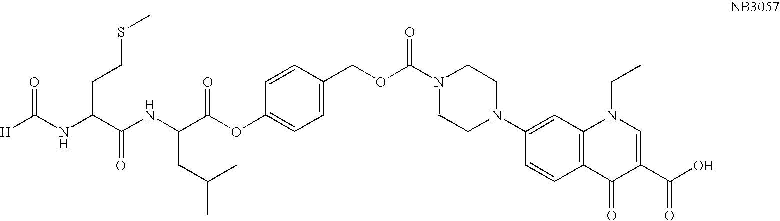

In one aspect, the compound has the structure:

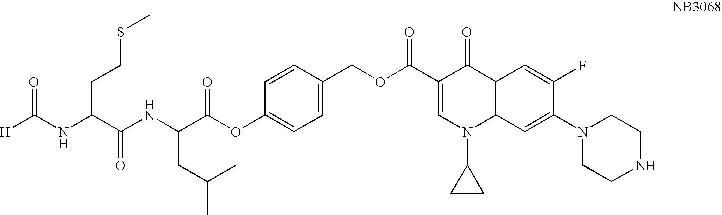

In one aspect, the compound has the structure:

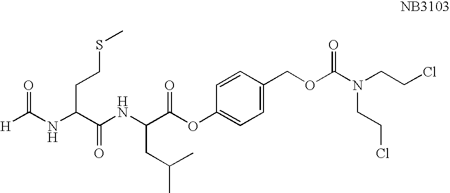

In one aspect, the compound has the structure:

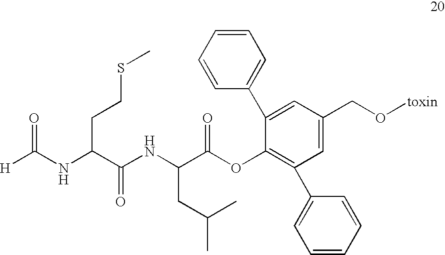

When toxin is absent, the compound is NB3145.

In one aspect, the compound has the structure:

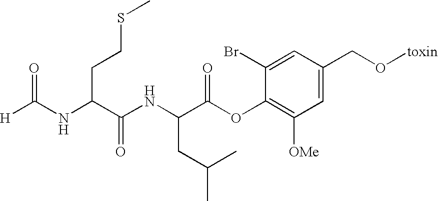

When toxin is absent, the compound is NB3162.

In one aspect, the compound has the structure:

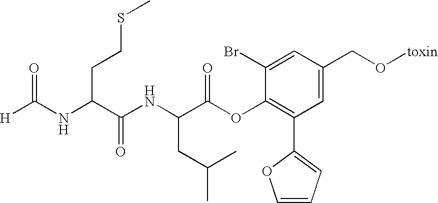

When toxin is absent, the compound is NB3177.

In one aspect, the compound has the structure:

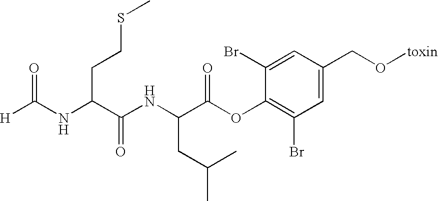

When toxin is absent, the compound is NB3144.

In one aspect, the compound has the structure:

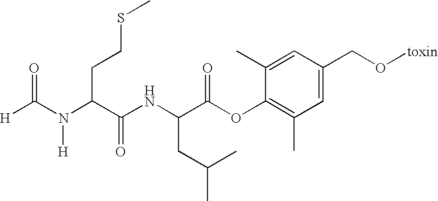

When toxin is absent, the compound is NB3165.

Also provided by this invention is a method for inhibiting the growth of a PDF expressing microorganism by contacting the microorganism with an effective amount of the compound as describe above. Methods to detect PDF expression are known in the art, see for example Wei and Pei (1997). This method is particularly useful in inhibiting the growth of gram-positive and gram-negative microorganisms, e.g., S. aureus, S. epidermidis, K pneumoniae, E. aerogenes, E. cloacae and those identified in Table 2, below. Further provided is a method for alleviating the symptoms of an infection in a subject, wherein the infection is caused by a PDF expressing microorganism, by administering or delivering to the subject an effective amount of the compound described above. Also provided by this invention is a method for treating an infection caused by a PDF expressing microorganism by administering or delivering to the subject an effective amount of the compound described above. A “subject” is defined above and includes mammals such as human patients. Examples of PDF expressing microorganims and the corresponding diseases and symptoms caused by infection by these microorganisms, are provided in Table 2, below.

TABLE 2

| | Disease or Symptom Caused by |
|---|---|
| PDF Expressing Microorganism | Infection |
| Gram-Positive | |
| Staphylococcus aureus | major human pathogen, |
| | bacteremia, pneumonia |
| Staphylococcus epidermidis and other | urinary tract infections, |
| coagulase-negative staphylococci | osteomyelitis, bacteremia |
| Streptococcus pyogenes | bacteremia, lymphagitis, |
| | pneumonia |
| Streptococcus pneumoniae | pneumonia, otitis media, sinusitis |
| Streptococcus agalactiae | primary bacteremia, pneumonia, |
| | endocarditis, osteomyelitis |
| Enterococcus species | urinary tract infections, |
| | bacteremia, endocarditis, intra- |
| | abdominal and pelvic infections, |
| | neonatal sepsis |
| Gram-Negative | |
| Neisseria gonorrhoeae | genital infection, perihepatitis |
| Moraxella catarrhalis | otitis media, lower respiratory |
| | tract infections, pneumonia, |
| | bacteremia |
| Campylobacter jejuni | acute enteritis, acute colitis, |
| | bacteremia |
| Enterobacteriaceae (including | enteric infections, urinary tract |
| Escherichia, Salmonella, Klebsiella, | infections, respiratory infections, |
| Enterobacter) | bacteremia |
| Pseudomonas aeruginosa | endocarditis, respiratory |
| | infections, bacteremia, central |
| | nervous system infections |
| Acinetobacter species | respiratory tract infections, |
| | bacteremia, genitourinary |
| Haemophilus influenzae | meningitis, epiglottitis, |
| | pneumonia, bacteremia |

This invention also provides a composition comprising the prodrug compounds as described above, alone or in combination with other compounds or other agents, known or yet to be discovered, and a carrier. In one embodiment, the carrier is a pharmaceutically acceptable carrier.

In the clinical use of the prodrug antibiotics will likely follow well established guidelines. Dosage will likely be similar to those already employed for most other antibiotics. It is estimated that a dose of prodrug will be in the range of 100 mg to 1 gm, given once every eight hours, or once a day, for one or two weeks, or until the patient tests negative for infectious organisms.

In one aspect, the invention encompasses a method of treating or protecting plants from infections caused by PDF expressing microorgansims by applying an effective amount of the substrate prodrug.

In order to achieve good dispersion and adhesion of the compounds as used to treat plants, it may be advantageous to formulate the compounds with components that aid dispersion and adhesion. Suitable formulations will be-known to those skilled in the art.

This invention also provides a method for treating or protecting plants from infection by microorganisms expressing PDF by applying an effective amount of the prodrug compound to the foliage, roots or the soil surrounding the plants or roots. These isolated compounds can be combined with known pesticides or insecticides.

Compounds within the present invention when used to treat or protect plants from infections caused by PDF expressing microorganisms, they can be formulated as wettable powders, granules and the like, or can be microencapsulated in a suitable medium and the like. Examples of other formulations include, but are not limited to soluble powders, wettable granules, dry flowables, aqueous flowables, wettable dispersible granules, emulsifiable concentrates and aqueous suspensions. Other suitable formulations will be known to those skilled in the art.

This invention further provides a method for administering the prodrug compound to fish in an amount effective to either prevent or treat an infection caused by PDF expressing microorganisms. The compound may be administered by incorporating the compound into the food supply for the fish. Alternatively, the compound may be added to the water in which fish live, or are contained within. Finally, the compound may be administered to the fish as a suitable pharmaceutical preparation. Other suitable formulations will be known to those skilled in the art.

Further provided is a process for producing the prodrugs of this invention. In general the process requires the following steps:

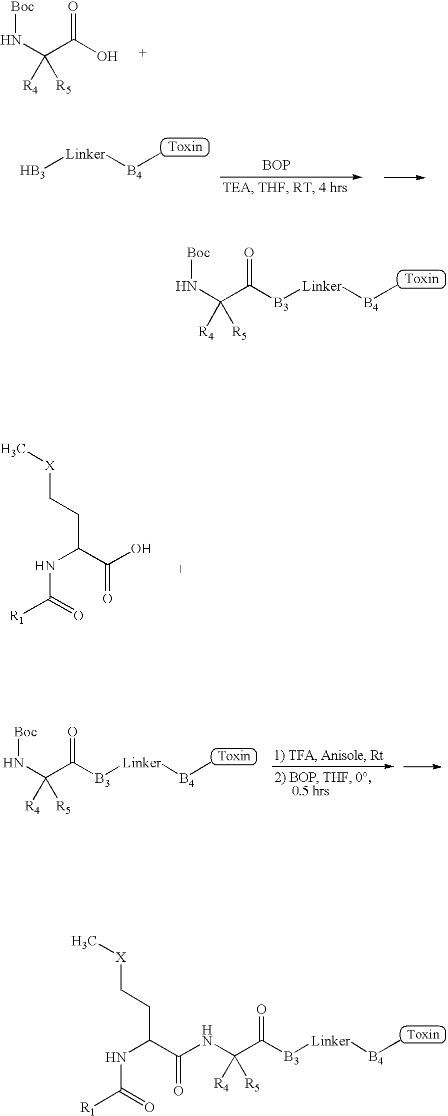

A rationally designed combinatorial library was used to select mechanism-based PDF inhibitors. Wei et al. (2000). The optimal inhibitor selected from the library possesses the structure: HS—CH₂—CH[(CH₂)₃—CH₃]—CONH—CH[—(CH₂)₃—NH—C(═NH)—NH₂]—CONH—R, where R is 2-naphthalene. This compound acts as a competitive PDF inhibitor with a Kᵢ of 15 nM.

With respect to the above diagram, X can be sulfur (methionine) or —CH₂-(norleucine). R₁ through R₅ and B₁ through B₃ are as defined above. Reaction conditions and full names for the abbreviations can be found in the experimental examples infra.

This invention provides a method for identifying potential therapeutic agents that inhibit the growth of an organism expressing PDF by contacting a sample containing the PDF expressing microorganism with an effective amount of a candidate prodrug compound. In a separate sample, the same microorganism is contacted with an effective amount of a prodrug of this invention. If the agent has comparable anti-proliferative ability as compared to a prodrug as described herein, the candidate is useful to useful to inhibit the growth or kill a PDF-expressing microorganism.

The prodrug is contacted with the sample under conditions that favor the activation of the prodrug by PDF and then assaying the sample growth inhibition or microbial death. Alternatively, the sample can be tested for the presence of the byproducts of the reaction of PDF on the substrate. Varying amounts of the substrate is contacted with a microorganism that expresses PDF for an amount of time effective for PDF to release the toxin from the cell, the bacteria is lysed and the analytes are analyzed using methods known in the art (e.g., HPLC) to identify the reaction products.

Varying concentrations of the potential agent are contacted with the sample to determine the optimal effective concentration of the agent. Thus, in one aspect, this invention relates to the discovery and use thereof of agents that are selective substrates for PDF.

Also provided by this invention are kits containing the prodrugs as described herein and instructions necessary to perform the screen.

The methods of the invention can be practiced in vitro, ex vivo or in vivo. In vivo practice of the invention in an animal such as a rat or mouse provides a convenient animal model system that can be used prior to clinical testing of the therapeutic agent or prodrug. In this system, a potential prodrug will be successful if microbial load is reduced or the symptoms of the infection are ameliorated, each as compared to an untreated, infected animal. It also can be useful to have a separate negative control group of cells or animals which has not been infected, which provides a basis for comparison.

When practiced in vivo, the candidate prodrug is administered or delivered to the animal in effective amounts. As used herein, the term “administering” for in vivo and ex vivo purposes means providing the subject with an effective amount of the candidate prodrug effective to reduce microbial load. In these instances, the agent or prodrug may be administered with a pharmaceutically acceptable carrier. The agents, prodrugs and compositions of the present invention can be used in the manufacture of medicaments and for the treatment of humans and other animals by administration in accordance with conventional procedures, such as an active ingredient in pharmaceutical compositions.

Methods of administering pharmaceutical compositions are known to those of ordinary skill in the art and include, but are not limited to, microinjection, intravenous or parenteral administration. The compositions are intended for topical, oral, or local administration as well as intravenously, subcutaneously, or intramuscularly. Administration can be effected continuously or intermittently throughout the course of the treatment. Methods of determining the most effective means and dosage of administration are known to those of skill in the art and will vary with the prodrug used for therapy, the purpose of the therapy, the microorganism being treated, the severity of the infection, and the subject being treated. Single or multiple administrations can be carried out with the dose level and pattern being selected by the treating physician. For example, the compositions can be administered to a subject already suffering from an antibiotic resistant bacterial infection. In this situation, an effective “therapeutic amount” of the composition is administered to prevent continued and to at least partially arrest microbial growth and proliferation and ameliorate the symptoms associated with an infection.

However, the prodrugs can be administered to subjects or individuals susceptible to or at risk of developing an infection. In these embodiments, a “prophylactically effective amount” of the composition is administered to maintain cell viability and function at a level near to the pre-infection level.

Administration in vivo can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be-carried out with the dose level and pattern being selected by the treating physician. Suitable dosage formulations and methods of administering the agents can be found below.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to an agent of the present invention, the pharmaceutical compositions can also contain other pharmaceutically active compounds or a plurality of compounds of the invention.

More particularly, an agent of the present invention also referred to herein as the active ingredient, may be administered for therapy by any suitable route including oral, rectal, nasal, topical (including transdermal, aerosol, buccal and sublingual), vaginal, parental (including subcutaneous, intramuscular, intravenous and intradermal) and pulmonary. It will also be appreciated that the route will vary with the condition and age of the recipient, and the disease being treated.

Ideally, the agent should be administered to achieve peak concentrations of the active compound at sites of disease. This may be achieved, for example, by the intravenous injection of the agent, optionally in saline, or orally administered, for example, as a tablet, capsule or syrup containing the active ingredient. Desirable blood levels of the agent may be maintained by a continuous infusion to provide a therapeutic amount of the active ingredient within disease tissue. The use of operative combinations is contemplated to provide therapeutic combinations requiring a lower total dosage of each component agent than may be required when each individual therapeutic compound or drug is used alone, thereby reducing adverse effects.

While it is possible for the agent to be administered alone, it is preferable to present it as a pharmaceutical formulation comprising at least one active ingredient, as defined above, together with one or more pharmaceutically acceptable carriers therefor and optionally other therapeutic agents. Each carrier must be “acceptable” in the sense of being compatible with the other ingredients of the formulation and not injurious to the patient.

Formulations include those suitable for oral, rectal, nasal, topical (including transdermal, buccal and sublingual), vaginal, parenteral (including subcutaneous, intramuscular, intravenous and intradermal) and pulmonary administration. The formulations may conveniently be presented in unit dosage form and may be prepared by any methods known in the art of pharmacy. Such methods include the step of bringing into association the active ingredient with the carrier that constitutes one or more accessory ingredients. In general, the formulations are prepared by uniformly and intimately bringing into association the active ingredient with liquid carriers or finely divided solid carriers or both, and then if necessary shaping the product.

Formulations of the present invention suitable for oral administration may be presented as discrete units such as capsules, cachets or tablets, each containing a predetermined amount of the active ingredient; as a powder or granules; a solution or suspension in an aqueous or non-aqueous liquid; or as an oil-in-water liquid emulsion or a water-in-oil liquid emulsion. The active ingredient may also be presented a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active-ingredient in a suitable liquid carrier.

Pharmaceutical compositions for topical administration according to the present invention may be formulated as an ointment, cream, suspension, lotion, powder, solution, past, gel, spray, aerosol or oil. Alternatively, a formulation may comprise a patch or a dressing such as a bandage or adhesive plaster impregnated with active ingredients and optionally one or more excipients or diluents.

If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound that enhances absorption or penetration of the agent through the skin or other affected areas. Examples of such dermal penetration enhancers include dimethylsulfoxide and related analogues.

The oily phase of the emulsions of this invention may be constituted from known ingredients in an known manner. While this phase may comprise merely an emulsifier (otherwise known as an emulgent), it desirably comprises a mixture of at lease one emulsifier with a fat or an oil or with both a fat and an oil. Preferably, a hydrophilic emulsifier is included together with a lipophilic emulsifier that acts as a stabilizer. In one variation, it includes both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulfate.

The choice of suitable oils or fats for the formulation is based on achieving the desired cosmetic properties, since the solubility of the active compound in most oils likely to be used in pharmaceutical emulsion formulations is very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the agent, such carriers as are known in the art to be appropriate.

Formulations suitable for nasal administration, wherein the carrier is a solid, include a coarse powder having a particle size, for example, in the range of about 20 to about 500 microns which is administered as a dry powder or in an inhaler device by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the agent.

Formulations suitable for parenteral administration include aqueous and non-aqueous isotonic sterile injection solutions which may contain anti-oxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Unit dosage formulations of interest include those containing a daily dose or unit, daily subdose, as herein above-recited, or an appropriate fraction thereof, of a agent.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies.

These agents of this invention and the above noted compounds and their derivatives may be used for the preparation of medicaments for use in the methods described herein.

In the clinical use of the prodrug antibiotics will likely follow established guidelines. Dosage will likely be similar to those already employed for most other antibiotics. It is estimated that a dose of prodrug will be in the range of 100 mg to 1 gm, given once every eight hours, or once a day, for one or two weeks, or until the patient tests negative for infectious organisms.

The following examples are intended to illustrate, but not limit the invention.

### EXAMPLE 1

### Synthetic Scheme for Compounds #1 and #2

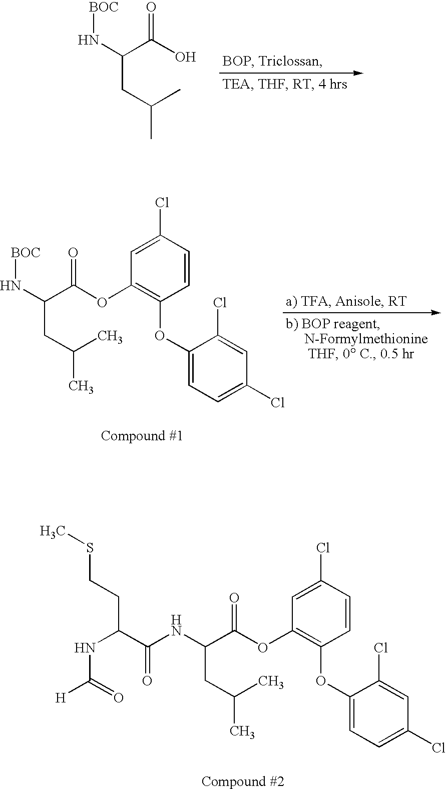 [0146] BOC—N-tert-butoxycarbonyl; BOP—benzotriazol-1-yloxytris(dimethylamino)phosphonium hexafluorophosphate; TEA—triethylamine; THF—tetrahydrofuran; RT—room temperature; TFA—trifluoroacetic acid

Synthesis of Compound 1:

A solution of N-Boc leucine (1.0 g, 4.32 mmol), triclosan (1.25 g, 4.32 mmol), benzotriazol-1-yloxytris(dimethylamino)phosphonium hexafluorophosphate (1.91 g, 4.32 mmol), and triethylamine (1.33 g, 12.9 mmol) in anhydrous THF (25 ml) was stirred at 0° C. under argon atmosphere for 4 hrs. Water (20 ml) was added and the reaction mixture was extracted with ethylacetate (2×30 ml). Combined organic layers was washed with water, brine, and dried over Na₂SO₄. Evaporation of the solvent and purification using silica gel column chromatography with 2% ethylacetate in hexane as eluant provided compound-1 as a colorless gum (1.66 g, 75%).

¹H NMR(CDCl₃, 500 MHz): 0.91 (d, 2H, J Hz), 1.43 (s, 9H), 1.51-1.60 (m, 2H), 1.69-1.73 (m, 1H), 4.43-4.48 (m, 1H), 4.83 (d, 1H, J=Hz), 6.80 (d, 1H, J=Hz), 6.86 (d, 1H, J=Hz), 7.14-7.26 (m, 2H), 7.26 (s, 1H), 7.43 (d, 1H, J=Hz).

Synthesis of Compound #2

A solution of compound-1 (0.25 g, 0.5 mmol), in anhydrous anisole (0.055 g, 0.5 mmol), was cooled to 0° C. and TFA (0.56 g, 5.0 mmol) was added slowly over 15 minutes. Ice bath was removed and stirring continued for another 3 hrs. All the volatiles were then removed under reduced pressure to get a gum. Anhydrous THF was added and cooled to 0° C. under argon atmosphere. Benzotriazol-1-yloxytris(dimethylamino)phosphonium hexafluorophosphate (0.25 g, 0.57 mmol), N-formyl methionine (0.1 g, 0.57 mmol), and triethylamine (0.21 g, 2.1 mmol) were added. Thin layer chromatography showed the completion of reaction after 0.5 hr at 0° C. The reaction was washed with water, brine, and dried Na₂SO₄. Purification on silica gel column chromatography provided Compound #2 as colorless thick gum.

¹H NMR (CDCl₃, 500 MHz): 0.88 (d, 3H, J=Hz), 0.91 (d, J=Hz), 1.507-1.56 (m, 1H), 1.60-1.71 (m, 2H), 1.95-2.02 (m, 1H), 2.09 (s, 3H), 2.50 (m, 1H), 2.58 (m, 1H), 4.65-4.70(m, 1H), 4.74 (q, 1H, J Hz), 6.48 (d, 1H), 6.78-6.85 (m, 2H), 7.15-7.25 (m, 3H), 7.44 (s, 1H), 8:17 (s, 1H).

### EXAMPLE 2

### General Synthetic Schemes

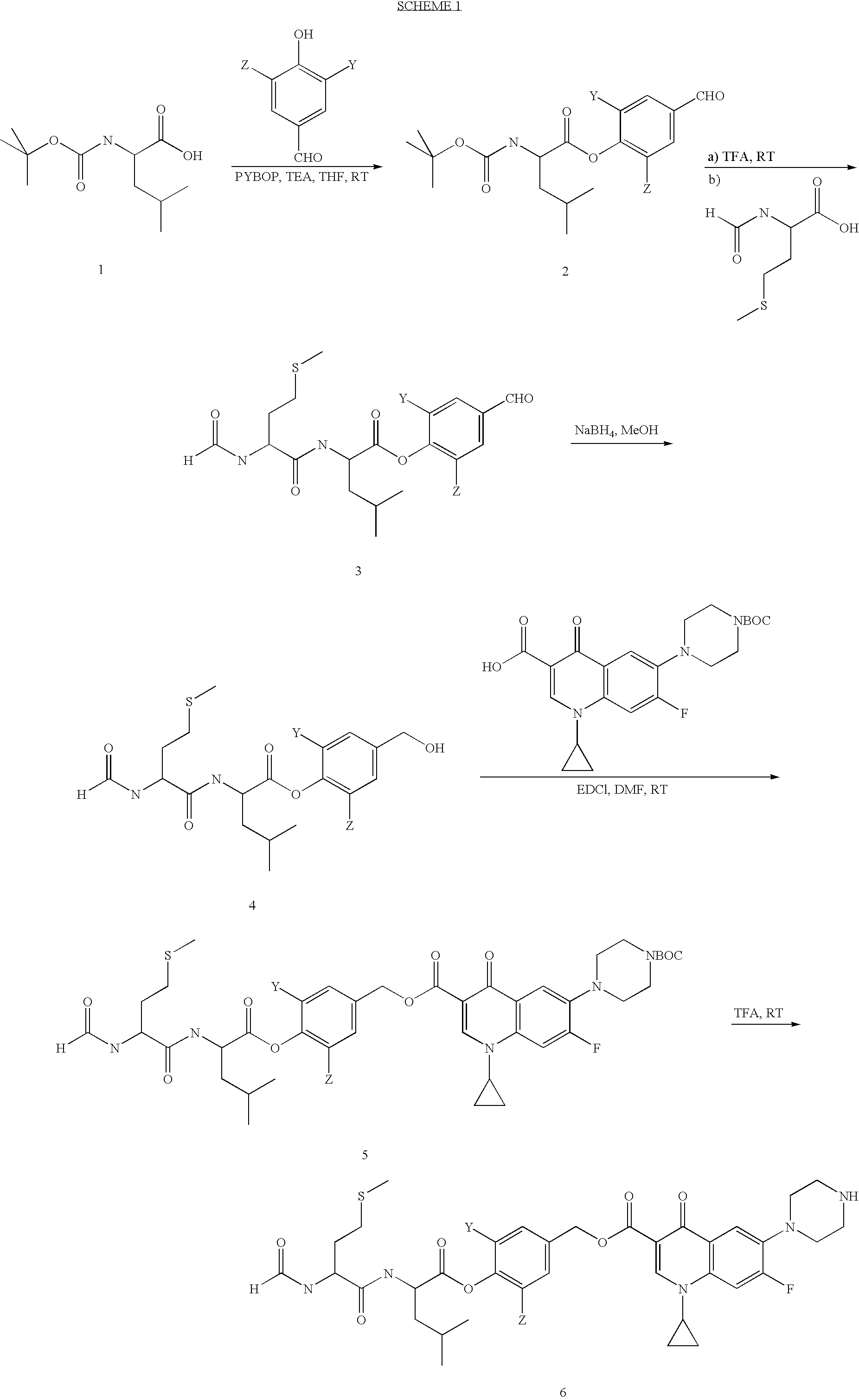 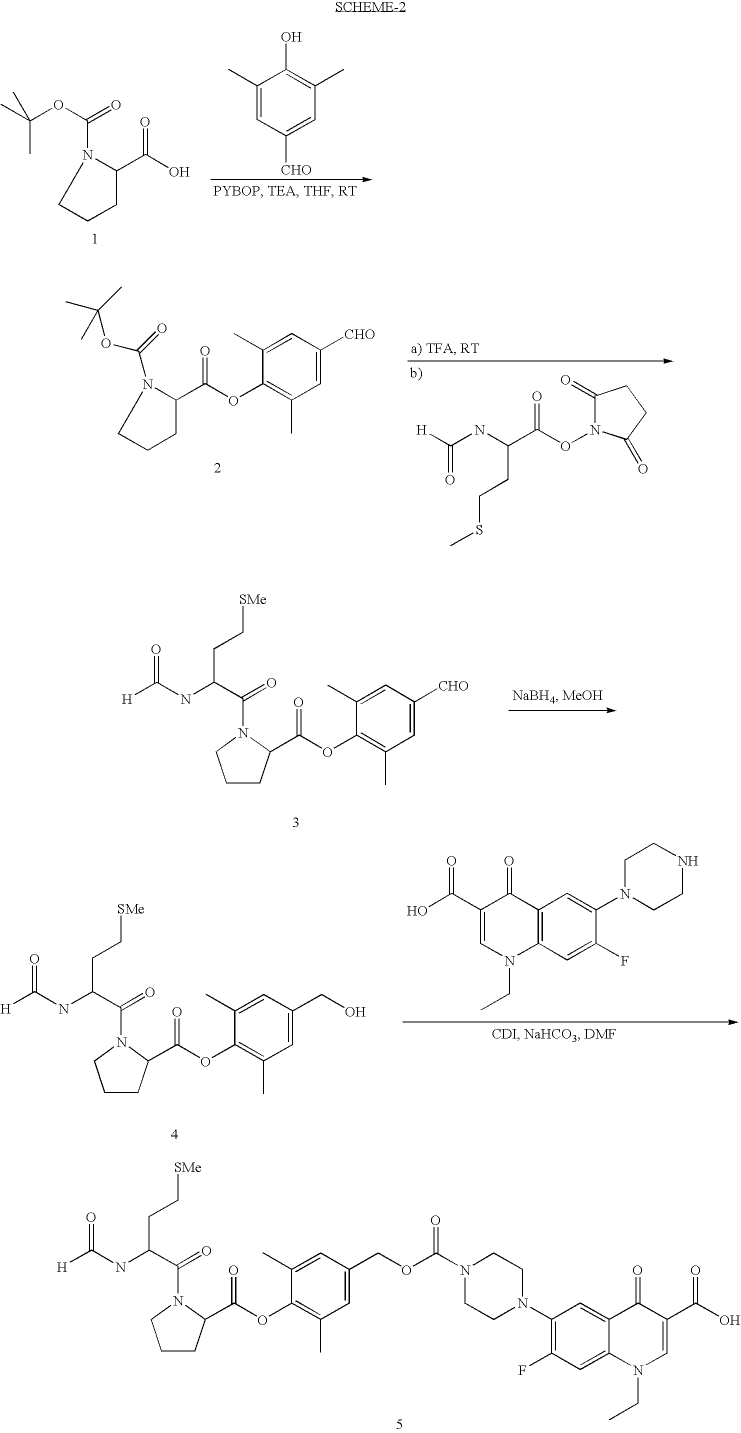 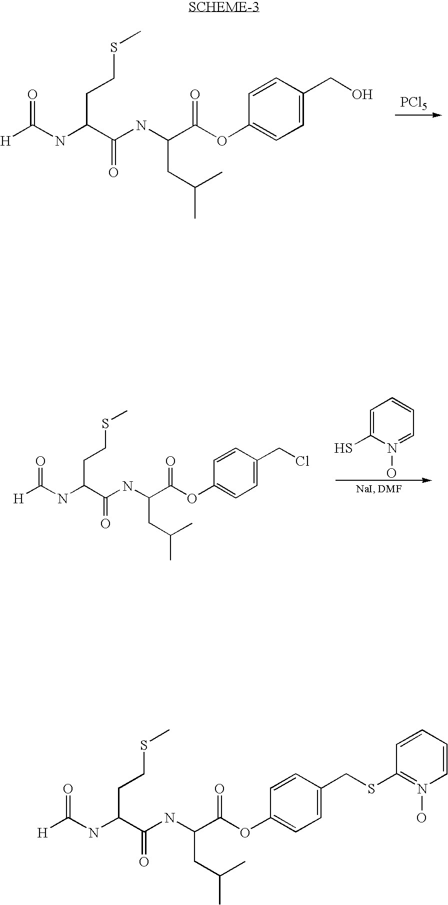 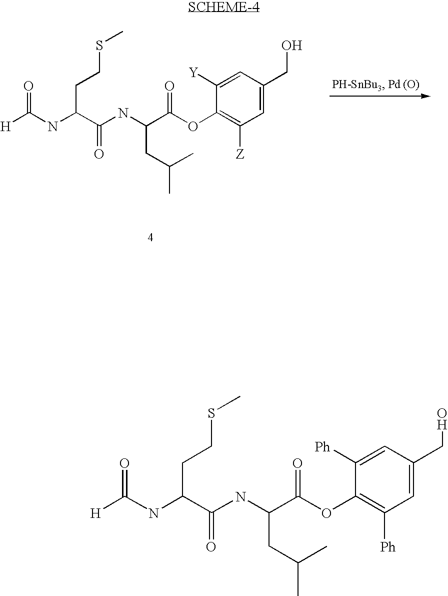 [0154] wherein in all the above synthetic schemes X and Y are independently the same or different and are selected from the group consisting of hydrogen, lower alkyl, substituted or unsubstituted, lower alkenyl, substituted or unsubstituted, lower alkynyl, substituted or unsubstituted, aryl groups, substituted or unsubstituted, heterocyclic groups, substituted or unsubstituted, lower alkoxy, lower alkylthio, halogen, cyano, nitro, carboxylate, sulfonate, alkyl sulfone, alkylsulfoxide and trialkylsilyl.

In the above general synthetic schemes and the following specific examples, the following applies: PyBOP is Benzotriazol-1-yl-oxytripyrrolidinophosphonium hexafluorophosphate; DMF is N,N-dimethylformamide; Na, CO₃ is sodium bicarbonate; RP-HPLC is reverse phase high performance liquid chromatography; TLC is thin layer chromatography; HCl is hydrochloric acid; TFA is trifluoroacetic acid; and DIEA is N,N-diisopropylethylamine.

Utilizing the above general methods, the following specific compounds were prepared.

### EXAMPLE 3

### Preparation of (r)-pyrrolidine-1,2-dicarboxylic acid 2-tert-butyl ester-1-4-formyl-2,6-dimethyl-phenyl) Ester (Scheme 2, Compound 2)

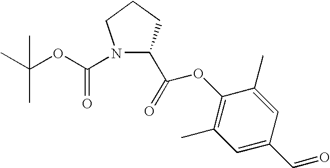

Py-BOP (6.8 g, 13.0 mmol) was added to a solution of N-tert-butyloxycarbonyl-D-proline (2.34 g, 10.9 mmol) and 3,5-dimethyl-4-hydroxybenzaldehyde (1.96 g, 13.0 mmol) in dry DMF (12 mL), and stirred to dissolve. N,N-Diisopropylethylamine (7.6 mL, 43.0 mmol) and 4-dimethylaminopyridine (122 mg, 1 mmol) were added with stirring. The resulting solution was stirred for 2.5 hours. The reaction mixture was covered with diethyl ether (100 mL) and washed with water, saturated aqueous sodium bicarbonate, and saturated brine. The ether layer was dried over anhydrous sodium sulfate, filtered, and concentrated under vacuum. The resulting red oil was purified by column chromatography on silica gel with dichloromethane eluant. Recovered clear, light yellow oil (1.16 g, 31%). ¹H NMR (CDCl₃, 500 MHz): 9.92 (s, 1H), 7.59 (s, 2H), 4.63-4.66 (m, 1H), 3.45-3.62 (m, 2H), 2.25 (s, 6H), 1.99-2.42 (m, 4H), 1.48 (s, 9H).

### EXAMPLE 4

### Preparation of 2-(s)-formylamino-4-methylsulfanyl-butyric Acid 2,5-dioxo-pyrrolidin-1-yl Ester (Intermediate in Scheme 2)

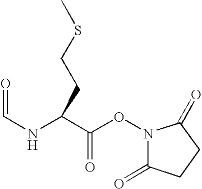

1,3-Dicyclohexycarbodiimide (2.48 g, 12.0 mmol) was added to an ice-cold solution of N-formyl-L-methionine (1.77 g, 10.0 mmol) and N-hydroxysuccinimide (1.38 g, 12.0 mmol) in dry THF (20 mL). The solution was stirred in an ice bath, and crystals formed quickly. The reaction was placed in a refrigerator overnight (about 14 hours). The crystalline precipitate (presumably dicyclohexylurea by-product) Was removed by filtration. The filtrate was diluted with methylene chloride, and the resulting solids were removed by filtration. The filtrate was reduced under vacuum to solids. These solids were dissolved in ethyl acetate, washed with saturated aqueous sodium bicarbonate and saturated brine. The ethyl acetate layer was dried over anhydrous sodium sulfate, filtered, and reduced to an oil under vacuum. The crude oil (2.8 g, 102% of theory) was used without further purification.

### EXAMPLE 5

### Preparation of 1-(s)-(2-formylamino-4-methylsulfanyl-butyryl)-pyrrolidine-2-carboxylic acid-4-formyl-2,6dimethyl-phenyl Ester (Scheme 2, Compound 3)

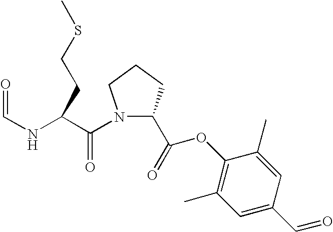

Trifluoroacetic acid (5.0 mL) was added to a solution of Compound 5 (1.16 g, 3.33 mmol) in dry dichloromethane (5.0 mL). The resulting solution was stirred under nitrogen for 30 minutes. The §olution-was concentrated under vacuum to remove excess TFA, and then re-dissolved in dichloromethane (7 mL). To this solution was added Compound 4 (0.91 g, 3.31 mmol) and DIEA (1.2 mL, 6.88 mmol). The reaction mixture was stirred at room temperature for 3 hours under nitrogen. The reaction mixture was taken up in ethyl acetate, then washed with aqueous HCl (0.1 M), saturated aqueous, sodium bicarbonate, and saturated brine. The ethyl acetate layer was dried over anhydrous sodium sulfate, filtered, and concentrated to dryness under vacuum. Recovered clear oil (1.09 g, 81% yield). ¹H NMR (CDCl3, 500 MHz): 9.91 (s, 1H), 8.18 (s, 1H), 7.58 (s, 2H), 6.49 (d, J=8.29 Hz, 1H), 5.08-5.11 (m, 1H), 4.78 (dd, J=3.5, 8.8 Hz, 1H), 3.91-3.94 (m, 1H), 3.69-3.73 (m, 1H); 2.53-2.58 (m, 2H), 2.41-2.46 (m, 1H), 2.25 (s, 6H), 2.15-2.29 (m, 2H), 2.11 (s, 3H), 2.08-2.16 (m, 2H), 1.89-1.95 (m, 1H).

### EXAMPLE 6

### Preparation of 1-(s)-(2-formylamino-4-methylsulfanyl-butyryl)-pyrrolidine-2-carboxylic acid-4-hydroxymethyl-2,6-dimethyl-phenyl Ester (Scheme 2, Compound 4)

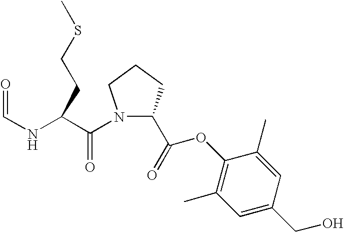

Sodium borohydride (50 mg, 1.3 mmol) was added to a solution of Compound 3 (1.08 g, 2.7 mmol) in anhydrous THF (10 mL). The resulting suspension was stirred for 20 minutes, after which TLC analysis indicated complete reduction of the aldehyde. The mixture was covered with ethyl acetate (100 mL) and quenched with aqueous HCl (0.1 M, 15 mL). The organic layer was separated and washed with aqueous HCl (0.1M, 15 mL), saturated aqueous NaHCO₃ (15 mL), and brine. The organic layer was dried over anhydrous sodium sulfate, filtered, and concentrated to dryness under vacuum. The residue was purified by silica gel column chromatography with ethyl acetate/dichloromethane eluent to afford white solid (165 mg, 15% yield). ¹H NMR (CDCl₃, 500 MHz): 8.19 (s, 1H), 7.07 (s, 2H), 6.44 (d, J=7.9 Hz, 1H), 5.05-5.10 (m, 1H), 4.84 (dd, J=4.9, 8.5 Hz, 1H), 4.61 (br.s, 2H), 3.84-3.88 (m, 1H), 3.80-3.84 (m, 1H), 2.54-2.57 (m, 2H), 2.43-2.48 (m, 1H), 2.18 (s, 6H), 2.06-2.46 (m, 5H), 2.05 (s, 3H), 1.89-1.94 (m, 1H).

### EXAMPLE 7

### Preparation of 1-ethyl-6-fluoro-7-(4-{4-[1-(s)-(2-formylamino-4-methylsulfanyl-butyryl)-pyrrolidine-2-carbonyloxy]-3,5-dimethyl-benzyloxycarbonyl}-piperazin-1-yl)-4-oxo-1,4-dihydro-quinoline-3-carboxylic Acid (Scheme 2, Compound 5):

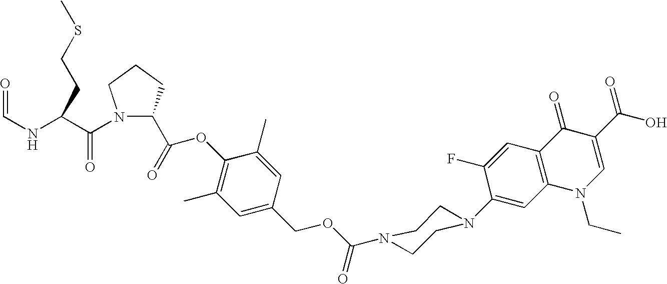

A solution of Compound 2 (20 mg, 0.049 mmol) and 1,1-carbonyldiimidazole (36 mg, 0.22 mmol in anhydrous DMF was stirred under argon for 3 hours. The resulting clear yellow solution was chilled in an ice bath, quenched with water (3 μL, 0.17 mmol), and stirred for 90 minutes. After warming to room temperature, norfloxacin (19 mg, 0.060 mmol) and sodium bicarbonate (17 mg, 0.20 mmol) were added to form a suspension. The suspension gradually (but not completely) cleared after stirring for 150 minutes. The reaction mixture was taken up in ethyl acetate (10 mL) and washed with 10% citric acid solution (2×4 mL) and saturated brine (4 mL). The ethyl acetate solution was dried over anhydrous magnesium sulfate, filtered, and reduced to dryness under vacuum. The resultant clear oil (29 mg) was purified by preparative RP-HPLC (20-60% acetonitrile), affording the product as a yellow powder (10.3 mg, 27% yield). 1H NMR (CDCl3, 500 MHz): 12.95 (br.s, 1H), 8.68 (s, 1H), 8;20 (s, 1H), 8.09 (d, J=12.5 Hz, 1H), 7.08 (s, 2H), 6.83 (d, J=6.57 Hz, 1H), 6.52 (d, J=8.3 Hz, 1H), 5.05-5.10 (m, 3H), 4.84 (dd, J=4.9, 8.5 Hz, 11H), 4.31 (q, J=7.2 Hz, 2H), 3.86-3.90 (m, 1H), 3.80-3.84 (m, 1H), 3.74 (br.s, 4H), 3.28 (br.s, 4H), 2.54-2.57 (m, 2H), 2.44-2.46 (m, 1H), 2.19 (br.s, 6H), 2.05 (s, 3H), 2.02-2.3 (m, 4H), 1.89-1.95 (m, 1H), 1.58 (t, J=7.2 Hz, 3H).

### EXAMPLE 8

### Preparation of 2-(2-formylamino-4-methylsulfanyl-butyrylamino)-4-ethyl-pentanoic Acid 4-chloromethyl-phenyl Ester (Scheme 3)

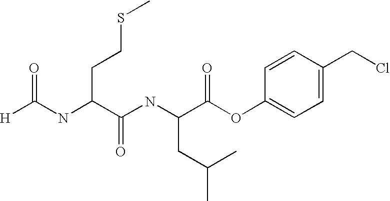

A solution of compound X (0.2 gr, 0.55 m mol) in anhydrous dichloromethane was cooled in an ice bath and PCl₅ (0.11 gr, 0.55 m mol) was added under argon atmosphere. After the completion of the reaction aqueous NaHCO₃ was added and stirred for 10 min. Organic layer was separated, washed with water, brine and dried (Na₂SO₄). Evaporation of volatiles provided compound XX which was used for the next reaction without further purification.

¹H NMR (CDCl₃, 500 MHz): 1.00-1.03 (m, 6H), 1.72-1.86 (m, 3H), 2.02-2.15 (m, 2H), 2.1 (s, 3H), 2.53-2.66 (m, 2H), 4.57 (s, 2H), 4.75-4.81 (m, 2H), 6.46-6.47 (m, 1H), 6.73-6.77 (m, 1H), 7.07-7.10 (m, 2H), 7.38-7.41 (m, 2H), 8.20 (s, 1H).

### EXAMPLE 9

### Preparation of 2-tert-Butoxycarbonylamino-4-methyl-pentanoic Acid 4-formyl-phenyl Ester (Scheme 1 Compound 2)

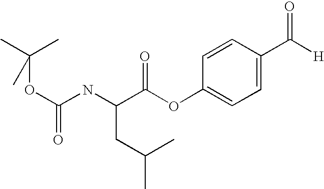

¹H NMR (CDCl₃, 500 MHz): 1.01-1.02 (m, 6H), 1.46 (s, 9H), 1.64-1.68 (m, 1H), 1.76-1.83 (m, 2H), 4.52-4.54 (m, 1H), 4.92-4.94 (d, 2H, J=7.6 Hz), 7.28-7.29 (d, 2H, J=8.48 Hz), 7.91-7.93 (d, 2H, J=8.48 Hz), 9.99 (s, 1H).

### EXAMPLE 10

### 2-(2-Formylamino-4-methylsulfanyl-butyrylamino)-4-methyl-pentanoic Acid 4-hydroxymethyl-phenyl Ester (Scheme 1, Compound 4)

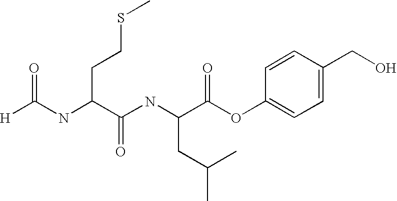

¹H NMR (CDCl₃, 500 MHz): 0.99-1.03 (m, 6H), 1.71-1.87 (m, 3H), 2.02-2.15 (m, 2H), 2.11 (s, 3H), 2.53-2.66 (m, 2H), 4.68 (s, 2H), 4.76-4.79 (m, 2H), 6.46-6.47 (m, 1H), 6.70-6.74 (m, 1H), 7.06-7.08 (m, 2H), 7.36-7.39 (m, 2H), 8.19 (s, 1H).

### EXAMPLE 11

### Preparation of 2-(2-Formylamino-4-methylsulfanyl-butyrylamino)-4-methyl-pentanoic acid 4-(1-hydroxy-1,2-dihydro-pyridin-2-ylsulfanylmethyl)-phenyl Ester (NB3024)

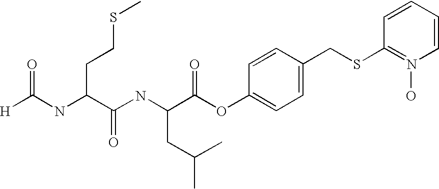

¹H NMR (CDCl₃, 500 MHz):0.98-1.02 (m, 6H), 1.71-1.84 (m, 3H), 2.02-2.21 (m, 2H), 2.14 (s, 3H), 2.60-2.66 (m, 2H), 4.15 (s, 2H), 4.75-4.78 (m, 2H), 6.48-6.50 (m, 1H), 6.70-6.74 (m, 1H), 7.06-7.08 (m, 311, 7.12 (d, 1H, 3=7.7 Hz), 7.21-7.24 (m, 1H) 7.36-7.39 (m, 2H), 8.19 (s, 1H), 8.26 (d, 1H, J=6.36 Hz).

### EXAMPLE 12

### Preparation of 2-(2-formylamino-4-methylsulfanyl-butyrylamino)-4-methyl-pentanoic Acid 2,6-dibromo-hydroxymethyl-phenyl Ester (NB3144)

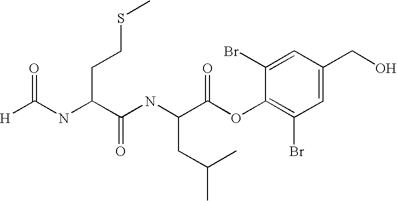

¹H NMR (CDCl₃, 500 MHz):0.99-1.03 (m, 6H), 1.71-1.87 (m, 3H), 2.02-2.15 (m, 2H), 2.11 (s, 3H), 2.53-2.66 (m, 2H), 4.68 (s, 2H), 4.76-4.79 (m, 2H), 6.41-6.46 (m, 1H), 6.61-6.63 (d, 1H, J=8.69), 7.58 (s, 2H), 8.21 (s, 1H).

### EXAMPLE 13

### Preparation of 2-(2-formylamino-4-methylsulfanyl-butyrylamino)-4-methyl-pentanoic Acid 5′-hydroxymethyl-[1,1′;3′,1″]terphenyl-2′-yl Ester (NB3145)

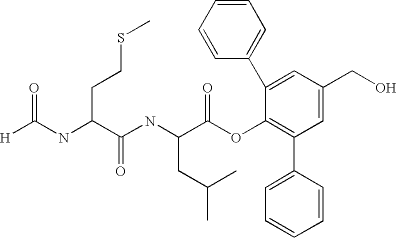

¹H NMR (CDCl₃, 500 MHz):0.70-0.75 (m, 6H), 1.71-1.87 (m, 3H), 2.01-2.16 (m, 2H), 2.13 (s, 3H), 2.53-2.66 (m, 2H), 4.45-4.53 (m, 2H), 4.78 (s, 2H), 6.08-6.10 (d, 1H), 6.27-6.27 (d, 1H, J=8.69), 7.33-7.40 (m, 12H), 8.12 (s, 1H).

### EXAMPLE 14

### Preparation of 2-(2-formylamino-4-methylsulfanyl-butyrylamino)-4-methyl-pentanoic Acid 2-bromo-4-hydroxymethyl-6-methoxy-phenyl Ester (NB3162)

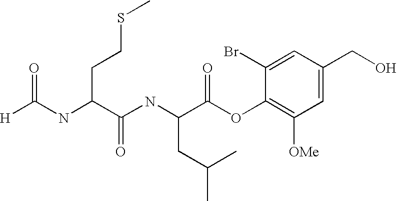

¹H NMR (CDCl₃, 500 MHz):1.00-1.02 (m, 6H), 1.60-1.77 (m, 2H), 1.80-1.86 (m, 1H), 1.95-2.12 (m, 2H), 2.11 (s, 3H), 2.61-2.66 (m, 2H), 3.82 (s, 3H), 4.66 (s, 2H), 4.74-4.78 (m, 1H), 4.93-4.98 (m, 1H), 6.41-6.45 (m, 1H), 6.60 (d, 1H, J=8.45), 6.94 (s, 1H), 7.16 (s, 1H), 8.20 (s, 1H).

### EXAMPLE 15

### Preparation of 2-(2-formylamino-4-methylsulfanyl-butyrylamino)-4-methyl-pentanoic Acid 4-hydroxymethyl-2,6-dimethyl-phenyl Ester (NB3165)

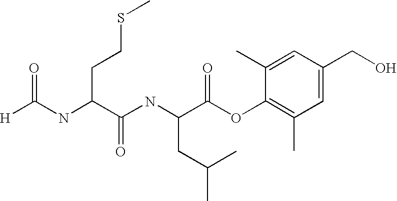

¹H NMR (CDCl₃, 500 MHz):1.02-1.03 (m, 6H), 1.72-1.93 (m, 3H), 2.01-2.17 (m, 2H), 2.11 (s, 3H), 2.15 (s, 6H), 2.65 (t, 2H, J=6.99 Hz), 4.62 (s, 2H), 4.76-4.86 (m, 2H), 6.40 (d, 1H, J=7.83 Hz), 6.65 (d, 1H, J=7.82 Hz), 7.09 (s, 2H), 8.20 (s, 1H)

### EXAMPLE 16

### Preparation of 1-ethyl-6-fluoro-7-(4-{4-[2-(2-formylamino-4-methylsulfanyl-butyrylamino)-4-methyl-pentanoyloxy]-benzyloxycarbonyl}-piperazin-1-yl)-4-oxo-1,4-dihydro-quinoline-3-carboxylic Acid (NB3057)

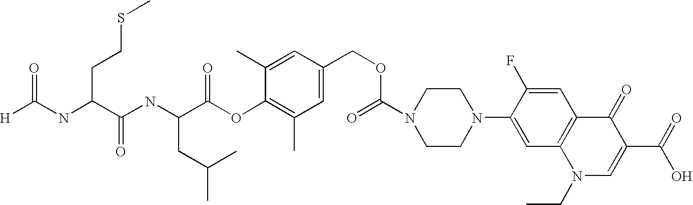

¹H NMR (CDCl₃, 500 MHz): 0.99-1.03 (m, 6H), 1.55 (m, 3H), 1.71-1.87 (m, 3H), 2.02-2.15 (m, 2H), 2.11 (s, 3H), 2.53-2.66 (m, 2H), 3.27 (brs, 4H), 3.78 (brs, 4H), 4.30 (q, 2H, J=7.19, 14.44 Hz), 4.76-4.80 (m, 2H), 5.15 (s, 2H), 6.46-6.47 (m, 1H), 6.71-6.72 (m, 1H), 6.83 (d, 1H, J=6.87 Hz), 7.08-7.10 (m, 2H), 7.38-7.41 (m, 2H), 8.10 (d, 1H, J=12.64 Hz), 8.21 (s, 1H), 8.68 (s, 1H).

### EXAMPLE 17

### Preparation of 1-cyclopropyl-6-fluoro-4-oxo-7-piperazin-1-yl-1,4-dihydro-quinoline-3-carboxylic Acid 4-[2-(2-formylamino-4-methylsulfanyl-butyrylamino)-4-methyl-pentanoyloxy]-benzyl Ester (NB3068)

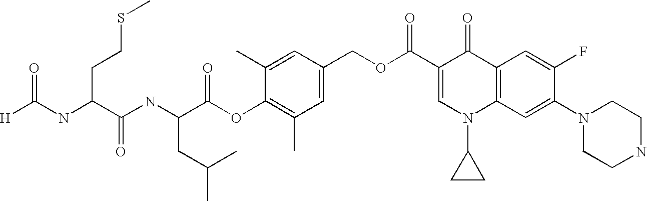

¹H NMR (DMSO-d6), 500 MHz): 0.89-0.91 (m, 3H0, 0.94-0.96 (m, 3H), 1.10-1.12 (m, 2H), 1.25-1.26 (m, 2H), 1.70-1.75 (m, 4H), 2.02-2.15 (m, 2H), 2.11 (s, 3H), 3.44 (brs, 4H), 3.78 (brs, 4H), 3.42-3.44 (m, 1H), 4.50-4.55 (m, 2H), 5.27 (s, 2H), 7.08-7.10 (m, 2H), 7.47-7.49 (m, 1H), 7.53-7.55 (m, 1H), 7.85 (d, 1H, J=12 HZ), 8.02 (s, 1H), 8.35(t, 1H, J=12.64 Hz), 8.50 (s, 1H), 8.62-8.68 (m, 1H), 8.79 (s, 2H).

### EXAMPLE 18

### Preparation of 2-(2-Formylamino-4-methylsulfanyl-butyrylamino)-4-methyl-pentanoic Acid 2-bromo-6furan-2-yl-4-hydroxymethyl-phenyl Ester (NB3177)

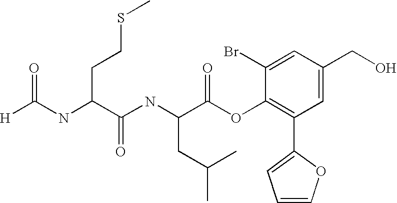

¹H NMR (CDCl₃, 500 MHz):0.93-1.02 (m, 6H), 1.75-1.87 (m, 3H), 2.04-2.15 (m, 2H), 2.17 (s, 3H), 2.50-2.56 (m, 2H), 4.71 (s, 2H), 4.71-4.77 (m, 2H), 5.04-5.08 (m, 1H), 6.39-6.43 (m, 1H), 6.49 (s, 1H), 6.62 (d, 1H, J=9.08 Hz), 6.71-6.80 (m, 1H), 7.50-7.55 (m, 2H), 7.71 (s, 1H), 8.21 (s, 1H).

### EXAMPLE 19

### Preparation of 2-(2-Formylamino-4-methylsulfanyl-butyrylamino)-4-methyl-pentanoic acid 4-{[bis-(2-chloro-ethyl)-carbamoyloxy]-methyl}-phenyl Ester (NB3103)

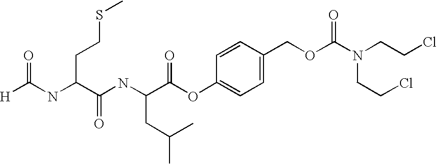 ¹H NMR (CDCl₃, 500 MHz):0.99-1.03 (m, 6H), 1.71-1.84 (m, 3H), 2.02-2.21 (m, 2H), 2.11 (s, 3H), 2.64-2.67 (m, 2H), 3.57-3.77 (m, 8H), 4.76-4.81 (m, 2H), 5.13 (s, 2H), 6.42-6.44 (m, 1H), 6.70 (d, 1H, J=8.1 Hz), 7.08-7.13 (m, 2H), 7.35-7.38 (m, 2H) 8.20 (s, 1H)

### EXAMPLE 20

### Susceptibility Testing

The NCCLS (National Committee for Clinical Laboratory Standards), method to determine MIC's of antimicrobial compounds is modified for high-throughput screening. All stocks of tested compounds are prepared in either water or in DMSO depending on solubility. At the highest concentration, DMSO content should not exceed 0.5%. Briefly, twenty 2-fold serial dilutions of test compounds from the highest concentration is made in a 384-well microtiter plate. Each well is inoculated with testing bacteria in broth to a final concentration of approximately 1-1.5×10⁶ cells/ml. Bacterial growth is determined by the increase of optical density at 600 nm using a microplate reader (Tecan SpectraFluor Plus). The MIC is defined as the lowest concentration at which bacterial growth (equivalent to visible growth) was inhibited after 16 to 18 hours of incubation at the appropriate temperature required for the bacteria growth. Results for Compound #2 are shown in Table 3 (bacteria) and Table 4.

TABLE 3

MIC (μg/ml)

| Organism | ATCC# | Exp. #1 | Exp. #2 |
|---|---|---|---|
| S. aureus | 700260 | ≦0.125 | ≦0.125 |
| S. aureus | 700698 | ≦0.125 | ≦0.125 |
| S. aureus | 700699 | 16 | 8 |
| S. aureus | 13301 | ≦0.125 | ≦0.125 |
| S. aureus | 11632 | ≦0.125 | ≦0.125 |
| S. aureus | 14154 | ≦0.125 | ≦0.125 |
| S. aureus | 700787 | ≦0.125 | ≦0.125 |
| S. aureus | 700788 | ≦0.125 | ≦0.125 |
| S. aureus | 700789 | ≦0.125 | ≦0.125 |
| S. aureus | 43300 | ≦0.125 | ≦0.125 |
| S. aureus | 33591 | ≦0.125 | ≦0.125 |
| S. aureus | 33592 | ≦0.125 | ≦0.125 |
| S. aureus | 33593 | ≦0.125 | ≦0.125 |
| S. epidermidis | 27626 | ≦0.125 | ≦0.125 |
| S. epidermidis | 700565 | 2 | 2 |
| S. epidermidis | 700566 | ≦0.125 | ≦0.125 |
| S. epidermidis | 700578 | 0.5 | 0.25 |
| S. epidermidis | 700583 | ≦0.125 | ≦0.125 |
| K. pneumoniae | 51503 | 8 | 8 |
| K. pneumoniae | 51504 | 1 | 2 |
| K. pneumoniae | 700721 | 2 | 2 |
| E. aerogenes | 29751 | 2 | 1 |
| E. cloacae | 23355 | 0.5 | 0.5 |
| E. aerogenes | 29009 | 0.25 | ≦0.125 |
| E. aerogenes | 13048 | 1 | 1 |
| E. aerogenes | 35028 | 4 | 2 |
| M. catarrhalis | 49265 | ≦0.125 | ≦0.125 |
| M. catarrhalis | 51584 | ≦0.125 | ≦0.125 |
| M. catarrhalis | 43627 | ≦0.125 | ≦0.125 |
| M. catarrhalis | 43628 | ≦0.125 | ≦0.125 |

TABLE 4

MIC (μg/ml)

| Organism | ATCC # | Exp. #1 | Exp. #2 |
|---|---|---|---|
| E. coli | | 16 | 16 |
| E. coli/Tem-1 | | 16 | 16 |
| MSSA | 700260 | ≦0.125 | 4 |
| MRSA | 700699 | 32 | 32 |
| MSSA | 33594 | ≦0.125 | ≦0.125 |
| MSSA | 11632 | 32 | 16 |
| E. faecalis | 49757 | 64 | 32 |
| E. faecalis | 700802 | 32 | 32 |
| E. fecium | | | |
| E. fecium | | | |
| E. aerogenes | 35028 | 32 | 16 |
| E. cloacae | 23355 | 16 | 1 |
| K. pneumoniae | 700721 | 4 | 8 |
| K. pneumoniae | 51503 | 4 | 8 |
| H. influenzae | 33533 | 64 | ≧64 |
| H. influenzae | 43334 | | |
| P. aeruginosa | 21726 | >64 | ≧64 |
| P. aeruginosa | 29872 | >64 | ≧64 |

E. coli/TEM—E. coli expressing TEM-1 beta-lactamase; MRSA—Methicillin Resistant S. Aureus; MSSA—Methicillin Sensitive S. Aureus.

Mammalian cells were treated with Compound #2 as described above. The compound is not toxic to mammalian cells (IC₅₀ of about 30 μM) after 16 hours of exposure.

Using the assay provided above, potency of Compound #2 was compared to triclosan. Results are shown in Table 5.

TABLE 5

| | | Triclosan |
|---|---|---|
| | Compound #2 | MIC, |
| MSSA (ATCC ##) | MIC, μg/ml | μg/ml |
| 700260 | 0.000031 | 0.000244 |
| 13301 | 0.000015 | 0.000488 |
| 11632 | 0.000977 | 0.001953 |
| 14154 | 0.000977 | 0.001953 |
| 33592 | 0.000488 | 0.003906 |
| 43300 | 0.000977 | 0.001953 |
| 700698 | 0.003906 | 0.003906 |
| 700699 | ≧4 | ≧4 |
| 700787 | 0.007813 | 0.001953 |
| 700788 | 0.062500 | 0.031250 |
| 700789 | 0.015630 | 0.015630 |
| 33591 | 0.015630 | 0.007813 |
| 33593 | 0.000488 | 0.000977 |

### EXAMPLE 21

### Activity of NB3057 and NB3068 Against Key Bacterial Pathogens

Table 6 compares the MIC of NB3057 and NB3068 with norfloxacin and ciprofloxacin against several bacterial pathogens.

TABLE 6

| | | MIC (μg/ml) | | | |
|---|---|---|---|---|---|
| | ATCC | | | | Cipro- |
| Organism | # | NB3057 | Norfloxacin | NB3068 | floxacin |
| E. coli | 25922 | 0.0625 | 0.0156 | <0.004 | 0.002 |
| E. faecalis | 29212 | 4 | 1 | 2 | 0.5 |
| S. aureus | 29213 | 0.125 | 0.5 | 2 | 0.25 |
| (MS) | | | | | |
| S. aureus | 33591 | 0.5 | 1 | not tested | not tested |
| (MR) | | | | | |
| P. aeruginosa | 27853 | 2 | 0.5 | 0.5 | 0.125 |

### EXAMPLE 22

### Measure of Plasma Stability of Various Compounds

Table 7 shows the plasma stability of several PDF ECTA compounds in PBS, Mueller Hinton Broth, Mouse Plasma and Human Plasma.

| | Stability | | | |
|---|---|---|---|---|
| | | Mueller | | Human |
| | | Hinton | Mouse | Plasma |
| | PBS, pH 7.4 | Broth | Plasma | t_{1/2}, |
| | t_{1/2}, | t_{1/2}, | t_{1/2}, | min (% left |
| | hrs (% left | hrs (% left | min (% left | after |
| Compound | after 6 hrs) | after 6 hrs) | after 10 min) | 120 min) |
| NB3057 | >12 (82%) | 10.0 | 1.4 | <0.5 |
| NB3068 | >12 (75%) | >12 (70%) | 0.6 | 4.1 |
| NB3144 | 3.3 | 2.9 | Not | Not |
| | | | determined | determined |
| NB3145 | >12 (99%) | >12 (99%) | >20 (94%) | >120 (94%) |
| NB3162 | >12 (98%) | >12 (91%) | 8.9 | 10.6 |
| NB3165 | >12 (85%) | >12 (95%) | >20 (94%) | >120 (94%) |
| NB3177 | >12 (95%) | >12 (89%) | >20 (88%) | 32.0 |

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

### REFERENCES

Apfel et al., Hydroxamic acid derivatives as potent peptide deformylase inhibitors and antibacterial agents. J. Med. Chem. 2000 43:2324-2331.
Apfel et al., Peptide Deformylase as an antibacterial drug target: assays for detection of its inhibition in Escherichia coli cell homogenates and intact cells. Anti. Agents and Chemo. (2001a) 45(4):1053-1057.
Apfel et al., Peptide Deformylase as an antibacterial drug target: target validation and resistance development. Anti. Agents and Chemo. (2001b) 45(4):1058-1064.
Becker A, et al., Iron center, substrate recognition and mechanism of peptide deformylase. Nat. Struct. Biol. 1998 December;5(12): 1053-8.
Chan, et al., Crystal structure of the Escherichia coli peptide deformylase. Biochemistry. 1997 Nov. 11;36(45):13904-9.
Chen, et al., Actinonin, a naturally occurring antibacterial agent, is a potent deformylase inhibitor. Biochemistry. 2000 Feb. 15;39(6):1256-62.
Clements, et al., Antibiotic Activity and characterization of BB-3497, a novel peptide deformylase inhibitor, Anti. Agents and Chemo. 45(2):563-570.
Durand, et al., Peptide Aldehyde Inhibitors of Bacterial Peptide Deformylases. Arch. Bio. And Biophysics. 367(2): 297-302.
Giglione C, et al., Peptide deformylase as a target for new generation, broad spectrum antimicrobial agents Mol Microbiol. (2000a) 36:1197205.
Giglione C, et al. Identification of eukaryotic peptide deformylases reveals universality of N-terminal protein processing mechanisms. EMBO J. (2000b) 19(21):5916-5929.
Hao B, et al., Structural basis for the design of antibiotics targeting peptide deformylase. Biochemistry. 1999 Apr 13;38(15):4712-9.
Hu Y J, et al., H-phosphonate derivatives as novel peptide deformylase inhibitors. Bioorg Med Chem Lett. 1998 Sep. 22;8(18):2479-82.
Huntington K M, et al., Synthesis and antibacterial activity of peptide deformylase inhibitors. Biochemistry. 2000 Apr. 18;39(15):4543-51.
Jayasekera M M, et al. Novel non-peptidic inhibitors of peptide deformylase Arch. Biochm. Biophys. 2000 Sep. 15;381(2):313-316.
Meinnel, T. Developing a rational strategy for new antibacterial agents. (1999) Pathol. Biol. 47: 780-783.
Meinnel T, et al., Methionine as translation start signal: a review of the enzymes of the pathway in Escherichia coli. Biochimie. 1993;75(12):1061-75. Review.
Nelson D L, Cox M M. Principles of Biochemistry. 2000, ed. Lehninger,
Rajagopalan P T, et al. Purification, characterization, and inhibition of peptide deformylase from Escherichia coli. Biochemistry. 1997 Nov. 11;36(45):13910-8.
Rajagopalan P T, Pei D. Oxygen-mediated inactivation of peptide deformylase. J. Biol. Chem. 1998 Aug. 28;273(35):22305-10.
Wei Y, and Pei D. Activation of antibacterial prodrugs by peptide deformylase. Bioorg Med Chem Lett. 2000 May 15;10(10):1073-6.
Wei Y. and Pei D. Continuous spectrophotometric assay of peptide deformylase. Anal Biochem. 1997 Jul. 15;250(1):29-34.
Wei Y, et al. Identification of a potent peptide deformylase inhibitor from a rationally designed combinatorial library. J Comb Chem. 2000 Nov.;2(6):650-7.

Patent Literature
U.S. Pat. No. 6,159,706 for “Application of Enzyme Prodrugs as Antiinfective-Agents”, issued Dec. 12, 2000.
PCT/US98/16607 for “Methods and Compositions For Overcoming Resistance to Biologic and Chemotherapy”.
PCT/US99/01332 for “Enzyme Catalyzed Therapeutic Agents”.
PCT/US00/20008 for “Enzyme Catalyzed Therapeutic Agent Compounds”.

1. A compound having the structure: 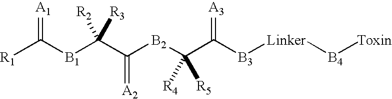
wherein R₁, R₂, R₄, and R₅ are independently the same or different and are selected from the group consisting of hydrogen, a substituted or unsubstituted C₅-C₁₄ aromatic or heteroaromatic (for example: phenylmethylene, 4-hydroxyphenylmethylene, imidazolemethylene, etc.); and a substituted or unsubstituted saturated or unsaturated C₁-C₆ alkyl (for example: methyl, ethyl, 3-hydroxypropyl, 3-aminopropyl, N-methyl-3-aminoethyl, 2-methoxyethyl, etc.);
wherein R₃ is selected from the group consisting of a substituted or unsubstituted aromatic or heteroaromatic (for example: phenylmethylene; triazolemethylene, thiophenemethylene, etc.), and a substituted or unsubstituted saturated or unsaturated C₁-C₆ alkyl (for example: ethyl, propyl, 2-hydroxyethyl, etc.) and —CH₂—CH₂—X—CH₃, wherein X is selected from the group consisting of O, S, NH, NR₆, and CH₂; where R₆ is a lower alkyl such as, for example, methyl or ethyl;
wherein A₁ and A₃ are independently the same or different and are selected from the group consisting of O, ═S, ═NH, ═N—OH, or ═N—R₇, where R₇ is hydrogen or a C₁-C₆ alkyl such as, for example, methyl, ethyl, or methoxymethyl;
wherein A₂ is selected from the group consisting of ═O, ═S; ═NH, ═N—OH, ═N—R₈, or ═C(R₉)(R₁₀), wherein R₈, R₉, and R₁₀ are independently the same or different and are selected from the group consisting of hydrogen or a C₁-C₆ alkyl such as, for example, methyl, ethyl, or methoxymethyl;
wherein B₁ is selected from the group consisting of —O—, —S—, —NH— or —N(R₁₁)—, wherein R₁₁ is selected from the group consisting of hydrogen and a C₁-C₆ alkyl such as, for example, methyl, ethyl, or methoxymethyl;
wherein B₂ is absent or is selected from the group consisting of —O—, —S—, —N(R₁₂), or —C(R₁₃)(R₁₄)—, where R₁₂, R₁₃, and R₁₄ are independently the same or different and are selected from the group consisting of hydrogen or a substituted or unsubstituted saturated or unsaturated C₁-C₆ alkyl (for example: methyl, ethyl, 3-hydroxypropyl, 3-aminopropyl, N-methyl-3-aminoethyl, 2-methoxyethyl, etc.), wherein when B₂ is —N(R₁₂)— or —C(R₁₃)(R₁₄)— it can be additionally joined through R₁₂, R₁₃ or R₁₄ to R₄ or R₅ to form a cyclic structure; wherein the fragment —B₂—C(R₄)(R₅)—C(═A₃)—in its entirety is proline or a proline derivative or analog,
wherein B₃ is absent or is selected from the group consisting of —O—, —S—, or —NH—, or —N(R₁₅)—, wherein R₁₅ is selected from the group consisting of hydrogen and a C₁-C₆ alkyl such as, for example, methyl, ethyl, or methoxymethyl;
wherein B₄ is absent or is selected from the group consisting of —O—, —S—, —N(R₆)—, and —C(R₁₆)(R₁₇) and wherein R₁₆ and R₁₇ are independently the same or different and are selected from the group consisting of hydrogen or a substituted or unsubstituted saturated or unsaturated C₁-C₆ alkyl such as, for example, methyl, ethyl, or methoxymethyl;
wherein a Linker is absent or is a traceless linker;
and wherein a toxin is an agent that is toxic upon activation by an activating enzyme with the proviso that the toxin is not 5-fluorodeoxyuridine, or any derivative or analog thereof.

2. The compound of claim 1, wherein R₁ and R₂ are both hydrogen.

3. The compound of claim 2, wherein R₃ is —CH₂—CH₂—X—CH₃, wherein X is selected from the group consisting of oxygen, sulfur or methyl.

4. The compound of claim 3, wherein X is sulfur- or oxygen.

5. The compound of claim 4, wherein A₁ and A₂ are both oxygen.

6. The compound of claim 5, wherein B₁ is —NH.

7. The compound of claim 1 wherein the linker is selected from the group consisting of C₆H₄—CH₂— and —C₆H₄—CH₂—X₁—C(═X₂)— wherein X₁ and X₂ are independently the same or different and are selected from the group consisting of —O—, —S —and —N(Rₐ), and where Rₐ is—hydrogen or a lower alkyl; and —(CH₂)ₙ—NR_{b}—(C═O)— wherein n=2 or 3 and R_{b} is hydrogen or a lower alkyl.

8. The compound of claim 7, wherein B₄ is absent.

9. The compound of claim 8, wherein the toxin is selected from the group consisting of 2-mercaptopyridine-N-oxide, ciprofloxacin, norfloxacin, nitrogen mustard and the derivatives, analogues and pharmaceutically acceptable salts thereof.

10. The compound of claim 9, wherein B₂ is —NH, B₃ is —O—, R₄ is 2-methyl-propyl and R₅ is hydrogen.

11. The compound of claim 9, wherein the toxin is norfloxacin or a derivative, analog or pharmaceutically acceptable salt thereof.

12. The compound of claim 1, wherein the compound is purified.

13. A composition comprising the compound of claim 1 and a carrier.

14. The composition of claim 13, wherein the carrier is a pharmaceutically acceptable carrier.

15. A method for inhibiting the growth of a microorganism, comprising contacting the microorganism with an effective amount of the compound of claim 1.

16. A method for treating a subject comprising administering to the subject an effective amount of the compound of claim 1.

17. A method for identifying potential therapeutic agents, comprising:
(a) contacting a microorganism with a compound of claim 1 under conditions that favor the incorporation of the compound into the microorganism; and
(b) assaying for amount of proliferation of microorganism in comparison to an untreated sample of the microorganism.
